SEQUENCE LISTING (38 sequences)

SEQ ID NO: 1 - DNA, 2622 bp, synthetic construct
  misc_feature 1..2622: note = BacCap1
  source 1..2622: mol_type = other DNA
tgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatctggcttcttttgttgatcacccacccgattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaatctagagcctgcagtctcgacaagctagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 2 - DNA, 2626 bp, synthetic construct
  misc_feature 1..2626: note = BacCap2
  source 1..2626: mol_type = other DNA
atcatggagataattaaaatgataaccatctcgcaaataaataagtattttactgttttcgtaacagttttgtaataaaaaaacctataaatagaccggagtagtcataccgtcccaccatcgggcgcggatcgtaccgggcccaagcttcctgttaagacggcttcttttgttgatcacccacccgattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaatctagagcctgcagtctcgacaagctagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 3 - DNA, 2580 bp, synthetic construct
  misc_feature 1..2580: note = BacCap3
  source 1..2580: mol_type = other DNA
atcatggagataattaaaatgataaccatctcgcaaataaataagtattttactgttttcgtaacagttttgtaataaaaaaacctataaatattccggattattcataccgtcccaccatcgggcgcggatcgtaccgggcccaagcttcctgttaagacggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaaaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 4 - DNA, 4123 bp, synthetic construct
  misc_feature 1..4123: note = BacRep1
  source 1..4123: mol_type = other DNA
gtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagtcgagattaccctgttatccctaccagtgtgttggatttattgttcaaagatacagtcatccaaatccacattaaccagatcgcaggcagtacaagcgtctggcacttttcccatgatatgatgaatatagcataatttttgatacgccttttttacgacagaaacgggttgagattctgacaccggaaagcattctaaacagtctttctggccgtgagtgaaacagatattactattctgattcattctctcacattgtctgcagggaaacaacattaagttcatgcctacgtgacgagaacatttgttttggtagcggtctgcgtagtttatcgaagcttccgcatctgacgtgcttggctgcgcaaccgattctctcactcgtttgggctcacttatatctgcatcactcggggcgggtcttttcttagcaccaccttttttgacgtaaaattcatgttccacttcaacaacgtgatcctttgcccaacgaaagaagtctttgacttcttgttttgttaccttgccaaaatcatgatccagtcggcgcgtcaattcaaatttgaacattcggtcttgcaacggttgttggtgttcgaatgtcgtactgttaccgtcaatcacggcgcacatgttcgtgttgcttgtaacgatcaccggtgtcgggtctatctgcgcagagcttttgcatttctggtctacgcgcactttgctgcctcctaaaattgctttggccgactccacgactttagcggtcattttgccttcctcccaccaaataaccatcttgtcgacacagtcgttgaatggaaagttctcattggtccagttaacgcagccataaaaaggtacagtgtgggctatggcctccgctatgtttgtttttcccgtagttgcaggtccaaacaaccaaatggtgtttcttttgccaaactttttcgtcgcccagcccaaaaatacggaagccgcatattgaggatcgtagccgtttaactccaaaatcttatagatgcgattgctggaaatgtcttccacgggttgctggcccaccaggtagtcgggggcggttttagtcaggctcataatcttgcccgcattgtccaaggcagctttgatttggctacgcgagttggatgccgcattaaacgagatgtatgaggcttgatcttcttgtatccattgcttctccgaggtaatacccttgtccaccaaccaaccgaccaattccatggcgaccgagatccgcgcccgatggtgggacggtatgaataatccggaatatttataggtttttttattacaaaactgttacgaaaacagtaaaatacttatttatttgcgagatggttatcattttaattatctccatgatctattaatattccggagtactgctagcaccatggatcccggtccgaagcgcgcggaattcaaaggcctacgtcgacgagctcactagtcgcggccgatctaataaacgataacgccgggtggcgtgaggcatgtaaaaggttacatcattatcttgttcgccatccggttggtataaatagacgttcatgttggtttttgtttcagttgcaagttggctgcggcgcgcgcagcacctttgcggccatctgcagaattcgcccttgttactcttcagccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 5 - DNA, 6697 bp, synthetic construct
  misc_feature 1..6697: note = DuoBac CapRep1
  source 1..6697: mol_type = other DNA
gtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagtcgagattaccctgttatccctaccagtgtgttggatttattgttcaaagatacagtcatccaaatccacattaaccagatcgcaggcagtacaagcgtctggcacttttcccatgatatgatgaatatagcataatttttgatacgccttttttacgacagaaacgggttgagattctgacaccggaaagcattctaaacagtctttctggccgtgagtgaaacagatattactattctgattcattctctcacattgtctgcagggaaacaacattaagttcatgcctacgtgacgagaacatttgttttggtagcggtctgcgtagtttatcgaagcttccgcatctgacgtgcttggctgcgcaaccgattctctcactcgtttgggctcacttatatctgcatcactcggggcgggtcttttcttagcaccaccttttttgacgtaaaattcatgttccacttcaacaacgtgatcctttgcccaacgaaagaagtctttgacttcttgttttgttaccttgccaaaatcatgatccagtcggcgcgtcaattcaaatttgaacattcggtcttgcaacggttgttggtgttcgaatgtcgtactgttaccgtcaatcacggcgcacatgttcgtgttgcttgtaacgatcaccggtgtcgggtctatctgcgcagagcttttgcatttctggtctacgcgcactttgctgcctcctaaaattgctttggccgactccacgactttagcggtcattttgccttcctcccaccaaataaccatcttgtcgacacagtcgttgaatggaaagttctcattggtccagttaacgcagccataaaaaggtacagtgtgggctatggcctccgctatgtttgtttttcccgtagttgcaggtccaaacaaccaaatggtgtttcttttgccaaactttttcgtcgcccagcccaaaaatacggaagccgcatattgaggatcgtagccgtttaactccaaaatcttatagatgcgattgctggaaatgtcttccacgggttgctggcccaccaggtagtcgggggcggttttagtcaggctcataatcttgcccgcattgtccaaggcagctttgatttggctacgcgagttggatgccgcattaaacgagatgtatgaggcttgatcttcttgtatccattgcttctccgaggtaatacccttgtccaccaaccaaccgaccaattccatggcgaccgagatccgcgcccgatggtgggacggtatgaataatccggaatatttataggtttttttattacaaaactgttacgaaaacagtaaaatacttatttatttgcgagatggttatcattttaattatctccatgatctattaatattccggagtactgctagcaccatggatcccggtccgaagcgcgcggaattcaaaggcctacgtcgacgagctcactagtcgcggccgatctaataaacgataacgccgggtggcgtgaggcatgtaaaaggttacatcattatcttgttcgccatccggttggtataaatagacgttcatgttggtttttgtttcagttgcaagttggctgcggcgcgcgcagcacctttgcggccatctgcagaattcgcccttgttactcttcagccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatctggcttcttttgttgatcacccacccgattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 6 - DNA, 6645 bp, synthetic construct
  misc_feature 1..6645: note = DuoBac CapRep2
  source 1..6645: mol_type = other DNA
gtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagtcgagattaccctgttatccctaccagtgtgttggatttattgttcaaagatacagtcatccaaatccacattaaccagatcgcaggcagtacaagcgtctggcacttttcccatgatatgatgaatatagcataatttttgatacgccttttttacgacagaaacgggttgagattctgacaccggaaagcattctaaacagtctttctggccgtgagtgaaacagatattactattctgattcattctctcacattgtctgcagggaaacaacattaagttcatgcctacgtgacgagaacatttgttttggtagcggtctgcgtagtttatcgaagcttccgcatctgacgtgcttggctgcgcaaccgattctctcactcgtttgggctcacttatatctgcatcactcggggcgggtcttttcttagcaccaccttttttgacgtaaaattcatgttccacttcaacaacgtgatcctttgcccaacgaaagaagtctttgacttcttgttttgttaccttgccaaaatcatgatccagtcggcgcgtcaattcaaatttgaacattcggtcttgcaacggttgttggtgttcgaatgtcgtactgttaccgtcaatcacggcgcacatgttcgtgttgcttgtaacgatcaccggtgtcgggtctatctgcgcagagcttttgcatttctggtctacgcgcactttgctgcctcctaaaattgctttggccgactccacgactttagcggtcattttgccttcctcccaccaaataaccatcttgtcgacacagtcgttgaatggaaagttctcattggtccagttaacgcagccataaaaaggtacagtgtgggctatggcctccgctatgtttgtttttcccgtagttgcaggtccaaacaaccaaatggtgtttcttttgccaaactttttcgtcgcccagcccaaaaatacggaagccgcatattgaggatcgtagccgtttaactccaaaatcttatagatgcgattgctggaaatgtcttccacgggttgctggcccaccaggtagtcgggggcggttttagtcaggctcataatcttgcccgcattgtccaaggcagctttgatttggctacgcgagttggatgccgcattaaacgagatgtatgaggcttgatcttcttgtatccattgcttctccgaggtaatacccttgtccaccaaccaaccgaccaattccatggcgaccgagatccgcgcccgatggtgggacggtatgaataatccggaatatttataggtttttttattacaaaactgttacgaaaacagtaaaatacttatttatttgcgagatggttatcattttaattatctccatgatctattaatattccggagtactgctagcaccatggatcccggtccgaagcgcgcggaattcaaaggcctacgtcgacgagctcactagtcgcggccgatctaataaacgataacgccgggtggcgtgaggcatgtaaaaggttacatcattatcttgttcgccatccggttggtataaatagacgttcatgttggtttttgtttcagttgcaagttggctgcggcgcgcgcagcacctttgcggccatctgcagaattcgcccttgttactcttcagccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcacctttaattcaacccaacacaatatattatagttaaataagaattattatcaaatcatttgtatattaattaaaatactatactgtaaattacattttatttctggcttcttttgttgatcacccacccgattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 7 - DNA, 6697 bp, synthetic construct
  misc_feature 1..6697: note = DuoBac CapRep3
  source 1..6697: mol_type = other DNA
gtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagtcgagattaccctgttatccctaccagtgtgttggatttattgttcaaagatacagtcatccaaatccacattaaccagatcgcaggcagtacaagcgtctggcacttttcccatgatatgatgaatatagcataatttttgatacgccttttttacgacagaaacgggttgagattctgacaccggaaagcattctaaacagtctttctggccgtgagtgaaacagatattactattctgattcattctctcacattgtctgcagggaaacaacattaagttcatgcctacgtgacgagaacatttgttttggtagcggtctgcgtagtttatcgaagcttccgcatctgacgtgcttggctgcgcaaccgattctctcactcgtttgggctcacttatatctgcatcactcggggcgggtcttttcttagcaccaccttttttgacgtaaaattcatgttccacttcaacaacgtgatcctttgcccaacgaaagaagtctttgacttcttgttttgttaccttgccaaaatcatgatccagtcggcgcgtcaattcaaatttgaacattcggtcttgcaacggttgttggtgttcgaatgtcgtactgttaccgtcaatcacggcgcacatgttcgtgttgcttgtaacgatcaccggtgtcgggtctatctgcgcagagcttttgcatttctggtctacgcgcactttgctgcctcctaaaattgctttggccgactccacgactttagcggtcattttgccttcctcccaccaaataaccatcttgtcgacacagtcgttgaatggaaagttctcattggtccagttaacgcagccataaaaaggtacagtgtgggctatggcctccgctatgtttgtttttcccgtagttgcaggtccaaacaaccaaatggtgtttcttttgccaaactttttcgtcgcccagcccaaaaatacggaagccgcatattgaggatcgtagccgtttaactccaaaatcttatagatgcgattgctggaaatgtcttccacgggttgctggcccaccaggtagtcgggggcggttttagtcaggctcataatcttgcccgcattgtccaaggcagctttgatttggctacgcgagttggatgccgcattaaacgagatgtatgaggcttgatcttcttgtatccattgcttctccgaggtaatacccttgtccaccaaccaaccgaccaattccatggcgaccgagatccgcgcccgatggtgggacggtatgaataatccggaatatttataggtttttttattacaaaactgttacgaaaacagtaaaatacttatttatttgcgagatggttatcattttaattatctccatgatctattaatattccggagtactgctagcaccatggatcccggtccgaagcgcgcggaattcaaaggcctacgtcgacgagctcactagtcgcggccgatctaataaacgataacgccgggtggcgtgaggcatgtaaaaggttacatcattatcttgttcgccatccggttggtataaatagacgttcatgttggtttttgtttcagttgcaagttggctgcggcgcgcgcagcacctttgcggccatctgcagaattcgcccttgttactcttcagccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatacggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 8 - DNA, 6645 bp, synthetic construct
  misc_feature 1..6645: note = DuoBac CapRep4
  source 1..6645: mol_type = other DNA
gtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagtcgagattaccctgttatccctaccagtgtgttggatttattgttcaaagatacagtcatccaaatccacattaaccagatcgcaggcagtacaagcgtctggcacttttcccatgatatgatgaatatagcataatttttgatacgccttttttacgacagaaacgggttgagattctgacaccggaaagcattctaaacagtctttctggccgtgagtgaaacagatattactattctgattcattctctcacattgtctgcagggaaacaacattaagttcatgcctacgtgacgagaacatttgttttggtagcggtctgcgtagtttatcgaagcttccgcatctgacgtgcttggctgcgcaaccgattctctcactcgtttgggctcacttatatctgcatcactcggggcgggtcttttcttagcaccaccttttttgacgtaaaattcatgttccacttcaacaacgtgatcctttgcccaacgaaagaagtctttgacttcttgttttgttaccttgccaaaatcatgatccagtcggcgcgtcaattcaaatttgaacattcggtcttgcaacggttgttggtgttcgaatgtcgtactgttaccgtcaatcacggcgcacatgttcgtgttgcttgtaacgatcaccggtgtcgggtctatctgcgcagagcttttgcatttctggtctacgcgcactttgctgcctcctaaaattgctttggccgactccacgactttagcggtcattttgccttcctcccaccaaataaccatcttgtcgacacagtcgttgaatggaaagttctcattggtccagttaacgcagccataaaaaggtacagtgtgggctatggcctccgctatgtttgtttttcccgtagttgcaggtccaaacaaccaaatggtgtttcttttgccaaactttttcgtcgcccagcccaaaaatacggaagccgcatattgaggatcgtagccgtttaactccaaaatcttatagatgcgattgctggaaatgtcttccacgggttgctggcccaccaggtagtcgggggcggttttagtcaggctcataatcttgcccgcattgtccaaggcagctttgatttggctacgcgagttggatgccgcattaaacgagatgtatgaggcttgatcttcttgtatccattgcttctccgaggtaatacccttgtccaccaaccaaccgaccaattccatggcgaccgagatccgcgcccgatggtgggacggtatgaataatccggaatatttataggtttttttattacaaaactgttacgaaaacagtaaaatacttatttatttgcgagatggttatcattttaattatctccatgatctattaatattccggagtactgctagcaccatggatcccggtccgaagcgcgcggaattcaaaggcctacgtcgacgagctcactagtcgcggccgatctaataaacgataacgccgggtggcgtgaggcatgtaaaaggttacatcattatcttgttcgccatccggttggtataaatagacgttcatgttggtttttgtttcagttgcaagttggctgcggcgcgcgcagcacctttgcggccatctgcagaattcgcccttgttactcttcagccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcacctttaattcaacccaacacaatatattatagttaaataagaattattatcaaatcatttgtatattaattaaaatactatactgtaaattacattttatttacggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 9 - DNA, 4518 bp, synthetic construct
  misc_feature 1..4518: note = DuoBac CapRep5
  source 1..4518: mol_type = other DNA
atcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcaaaggcctacgtcgacgagctcactagtaacggccgccagtgtgctggaattcgcccttcgcggatcctgttaagacggcggggttctacgagattgtgattaaggtccccagcgaccttgacgagcatctgcccggcatttctgacagctttgtgaactgggtggccgagaaggaatgggagttgccgccagattctgacgtggatctgaatctgattgagcaggcacccctgaccgtggccgagaagctgcagcgcgactttctgacggaatggcgccgtgtgagtaaggccccggaggcccttttctatgtgcaatttgagaagggagagagctacttccacatgcacgtgctcgtggaaaccaccggggtgaaatccatggttttgggacgtttcctgagtcagattcgcgaaaaactgattcagagattttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagtaagtctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcacctttaattcaacccaacacaatatattatagttaaataagaattattatcaaatcatttgtatattaattaaaatactatactgtaaattacattttatttacggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 10 - DNA, 6596 bp, synthetic construct
  misc_feature 1..6596: note = DuoBac CapRep6
  source 1..6596: mol_type = other DNA
atttattgttcaaagatacagtcatccaaatccacattaaccagatcgcaggcagtacaagcgtctggcacttttcccatgatatgatgaatatagcataatttttgatacgccttttttacgacagaaacgggttgagattctgacaccggaaagcattctaaacagtctttctggccgtgagtgaaacagatattactattctgattcattctctcacattgtctgcagggaaacaacattaagttcatgcctacgtgacgagaacatttgttttggtagcggtctgcgtagtttatcgaagcttccgcatctgacgtgcttggctgcgcaaccgattctctcactcgtttgggctcacttatatctgcatcactcggggcgggtcttttcttagcaccaccttttttgacgtaaaattcatgttccacttcaacaacgtgatcctttgcccaacgaaagaagtctttgacttcttgttttgttaccttgccaaaatcatgatccagtcggcgcgtcaattcaaatttgaacattcggtcttgcaacggttgttggtgttcgaatgtcgtactgttaccgtcaatcacggcgcacatgttcgtgttgcttgtaacgatcaccggtgtcgggtctatctgcgcagagcttttgcatttctggtctacgcgcactttgctgcctcctaaaattgctttggccgactccacgactttagcggtcattttgccttcctcccaccaaataaccatcttgtcgacacagtcgttgaatggaaagttctcattggtccagttaacgcagccataaaaaggtacagtgtgggctatggcctccgctatgtttgtttttcccgtagttgcaggtccaaacaaccaaatggtgtttcttttgccaaactttttcgtcgcccagcccaaaaatacggaagccgcatattgaggatcgtagccgtttaactccaaaatcttatagatgcgattgctggaaatgtcttccacgggttgctggcccaccaggtagtcgggggcggttttagtcaggctcataatcttgcccgcattgtccaaggcagctttgatttggctacgcgagttggatgccgcattaaacgagatgtatgaggcttgatcttcttgtatccattgcttctccgaggtaatacccttgtccaccaaccaaccgaccaattccatggcgaccgagatccgcgcccgatggtgggacggtatgaataatccggaatatttataggtttttttattacaaaactgttacgaaaacagtaaaatacttatttatttgcgagatggttatcattttaattatctccatgatctattaatattccggagtactgctagcaccatggatcccggtccgaagcgcgcggaattcaaaggcctacgtcgacgagctcactagtcgcggccgatctaataaacgataacgccgggtggcgtgaggcatgtaaaaggttacatcattatcttgttcgccatccggttggtataaatagacgttcatgttggtttttgtttcagttgcaagttggctgcggcgcgcgcagcacctttgcggccatctgcagaattcgcccttgttactcttcagccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgtaatgagacgcacaaactaatatcacaaactggaaatgtctatcaatatatagttgctgatgtaccgcagcatgctatgcatcagctgctagtactccggaatattaatagatcatggagataattaaaatgataaccatctcgcaaataaataagtattttactgttttcgtaacagttttgtaataaaaaaacctataaatagaccggagtagtcataccgtcccaccatcgggcgcggatcgtaccgggcccaagcttcctgttaagacggcttcttttgttgatcacccacccgattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 11 - DNA, 6645 bp, synthetic construct
  misc_feature 1..6645: note = DuoBac CapRep7
  source 1..6645: mol_type = other DNA
gtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagtcgagattaccctgttatccctaccagtgtgttggatttattgttcaaagatacagtcatccaaatccacattaaccagatcgcaggcagtacaagcgtctggcacttttcccatgatatgatgaatatagcataatttttgatacgccttttttacgacagaaacgggttgagattctgacaccggaaagcattctaaacagtctttctggccgtgagtgaaacagatattactattctgattcattctctcacattgtctgcagggaaacaacattaagttcatgcctacgtgacgagaacatttgttttggtagcggtctgcgtagtttatcgaagcttccgcatctgacgtgcttggctgcgcaaccgattctctcactcgtttgggctcacttatatctgcatcactcggggcgggtcttttcttagcaccaccttttttgacgtaaaattcatgttccacttcaacaacgtgatcctttgcccaacgaaagaagtctttgacttcttgttttgttaccttgccaaaatcatgatccagtcggcgcgtcaattcaaatttgaacattcggtcttgcaacggttgttggtgttcgaatgtcgtactgttaccgtcaatcacggcgcacatgttcgtgttgcttgtaacgatcaccggtgtcgggtctatctgcgcagagcttttgcatttctggtctacgcgcactttgctgcctcctaaaattgctttggccgactccacgactttagcggtcattttgccttcctcccaccaaataaccatcttgtcgacacagtcgttgaatggaaagttctcattggtccagttaacgcagccataaaaaggtacagtgtgggctatggcctccgctatgtttgtttttcccgtagttgcaggtccaaacaaccaaatggtgtttcttttgccaaactttttcgtcgcccagcccaaaaatacggaagccgcatattgaggatcgtagccgtttaactccaaaatcttatagatgcgattgctggaaatgtcttccacgggttgctggcccaccaggtagtcgggggcggttttagtcaggctcataatcttgcccgcattgtccaaggcagctttgatttggctacgcgagttggatgccgcattaaacgagatgtatgaggcttgatcttcttgtatccattgcttctccgaggtaatacccttgtccaccaaccaaccgaccaattccatggcgaccgagatccgcgcccgatggtgggacggtatgaataatccggaatatttataggtttttttattacaaaactgttacgaaaacagtaaaatacttatttatttgcgagatggttatcattttaattatctccatgatctattaatattccggagtactgctagcaccatggatcccggtccgaagcgcgcggaattcaaaggcctacgtcgacgagctcactagtcgcggccgatctaataaacgataacgccgggtggcgtgaggcatgtaaaaggttacatcattatcttgttcgccatccggttggtataaatagacgttcatgttggtttttgtttcagttgcaagttggctgcggcgcgcgcagcacctttgcggccatctgcagaattcgcccttgttactcttcagccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcacctttaattcaacccaacacaatatattatagttaaataagaattattatcaaatcatttgtatattaattaaaatactatactgtaaattacattcgatgcatggtaagctttgttgatcacccacccgattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 12 - DNA, 5142 bp, synthetic construct
  misc_feature 1..5142: note = DuoBac CapTrans1
  source 1..5142: mol_type = other DNA
atcatggagataattaaaatgataaccatctcgcaaataaataagtattttactgttttcgtaacagttttgtaataaaaaaacctataaatagaccggagtagtcataccgtcccaccatcgggcgcggatcgtaccgggcccaagcttcctgttaagacggcttcttttgttgatcacccacccgattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaatctagagcctgcagtctcgacaagctagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggggggtaccagatcccatgggagctctgcagaattctctagaggcctcgcgagatcgatctagaaagcttcccggggggatctgggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctggaggggtggagtcgtgacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgaattctagaccaccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctcgatgctttatttgtgaaatttgtgatgctattgctttatttgtaaccattataagctgcaataaacaagttaacaacaacaattgcattcattttatgtttcaggttcagggggaggtgtgggaggttttttaaactaggtcacgactccacccctccaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccca

SEQ ID NO: 13 - DNA, 3111 bp, synthetic construct
  misc_feature 1..3111: note = BacTrans1
  source 1..3111: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgataatacgactcactatagggagacccaagcttgatcccccctcttcctcctcctcaagggaaagctgcccacttctagctgccctgccatcccctttaaagggcgacttgctcagcgccaaaccgcggctccagccctctccagcctccggctcagccggctcatcagtcggtcaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttgtccgtgttgcttggtcttcacctgtgcagaattgcgaaccatggattcatcgacggtaccgcgggccctcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcccc

SEQ ID NO: 14 - DNA, 2412 bp, synthetic construct
  misc_feature 1..2412: note = BacTrans2
  source 1..2412: mol_type = other DNA
gggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctggaggggtggagtcgtgacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgaattctagaccaccatgcagagggtgaacatgatcatggctgagagccctggcctgatcaccatctgcctgctgggctacctgctgtctgctgagtgcactgtgttcctggaccatgagaatgccaacaagatcctgaacaggcccaagagatacaactctggcaagctggaggagtttgtgcagggcaacctggagagggagtgcatggaggagaagtgcagctttgaggaggccagggaggtgtttgagaacactgagaggaccactgagttctggaagcagtatgtggatggggaccagtgtgagagcaacccctgcctgaatgggggcagctgcaaggatgacatcaacagctatgagtgctggtgcccctttggctttgagggcaagaactgtgagctggatgtgacctgcaacatcaagaatggcagatgtgagcagttctgcaagaactctgctgacaacaaggtggtgtgcagctgcactgagggctacaggctggctgagaaccagaagagctgtgagcctgctgtgccattcccatgtggcagagtgtctgtgagccagaccagcaagctgaccagggctgaggctgtgttccctgatgtggactatgtgaacagcactgaggctgaaaccatcctggacaacatcacccagagcacccagagcttcaatgacttcaccagggtggtggggggggaggatgccaagcctggccagttcccctggcaagtggtgctgaatggcaaggtggatgccttctgtgggggcagcattgtgaatgagaagtggattgtgactgctgcccactgtgtggagactggggtgaagatcactgtggtggctggggagcacaacattgaggagactgagcacactgagcagaagaggaatgtgatcaggatcatcccccaccacaactacaatgctgccatcaacaagtacaaccatgacattgccctgctggagctggatgagcccctggtgctgaacagctatgtgacccccatctgcattgctgacaaggagtacaccaacatcttcctgaagtttggctctggctatgtgtctggctggggcagggtgttccacaagggcaggtctgccctggtgctgcagtacctgagggtgcccctggtggacagggccacctgcctgaggagcaccaagttcaccatctacaacaacatgttctgtgctggcttccatgaggggggcagggacagctgccagggggactctgggggcccccatgtgactgaggtggagggcaccagcttcctgactggcatcatcagctggggggaggagtgtgccatgaagggcaagtatggcatctacaccaaagtctccagatatgtgaactggatcaaggagaagaccaagctgacctgactcgatgctttatttgtgaaatttgtgatgctattgctttatttgtaaccattataagctgcaataaacaagttaacaacaacaattgcattcattttatgtttcaggttcagggggaggtgtgggaggttttttaaactaggtcacgactccacccctccaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccc

SEQ ID NO: 15 - DNA, 2844 bp, synthetic construct
  misc_feature 1..2844: note = BacTrans3
  source 1..2844: mol_type = other DNA
ggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatcagcttgcatgcaggcctctgcagtcgacgggcccgtcgatcttgatgcaacttaattttattaggacaaggctggtgggcactggagtggcaacttccagggccaggagaggcactggggaggggtcacagggatgccacgggcggccgctcgagatctggatccagcgccttggcctctgaaagtggtgggattacaggcgtgagccactgtgcctggcttatctttatttctttacaacaggaaagagaaaatgtatctattccctcccctacccccaatcccacgcccccacccctgccttgtttgagctggagtctcccttccagtagtctgcttcagggtcctgagttctcttcctggcacgtttnnnnnnnnnnnnnnnnnnnnnnnnnnnnnnnnnnnnnnnnnnnnnnnnnnnnnnnnnnnnnnnnnnnnnnnntgccctggcagtcagtaggttgtgacaggctgagcagagagcttcttgggcttgcagcatctctcctgtcctccttgtcaggctccagagctgggggtgcccggactagtacatcatctatactgtagtgtctcatcgcaaacttacagtatatgatgaaatcccagccagggccccactgggggcacaggaagcatagcgccgatatctagatgcattcgcgaggtaccgagctcgaattcgcccttaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaggtaccgaattaagggcgaattcactggccgtcgttttacaacgtcgtgactgggaaaaccctggcgttacccaacttaatcgccttgcagcacatccccctttcgccagctggcgtaatagcgaagaggcccgcaccggatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccc

SEQ ID NO: 16 - DNA, 2414 bp, synthetic construct
  misc_feature 1..2414: note = BacTrans4
  source 1..2414: mol_type = other DNA
tgggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctggaggggtggagtcgtgacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgaattctagaccaccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctcgatgctttatttgtgaaatttgtgatgctattgctttatttgtaaccattataagctgcaataaacaagttaacaacaacaattgcattcattttatgtttcaggttcagggggaggtgtgggaggttttttaaactaggtcacgactccacccctccaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccca

SEQ ID NO: 17 - DNA, 2798 bp, synthetic construct
  misc_feature 1..2798: note = BacTrans5
  source 1..2798: mol_type = other DNA
ggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatcagcttgcatgcaggcctctgcagtcgacgggcccgtcgatcttgatgcaacttaattttattaggacaaggctggtgggcactggagtggcaacttccagggccaggagaggcactggggaggggtcacagggatgccacgggcggccgctcgagatctggatccagcgccttggcctctgaaagtggtgggattacaggcgtgagccactgtgcctggcttatctttatttctttacaacaggaaagagaaaatgtatctattccctcccctacccccaatcccacgcccccacccctgccttgtttgagctggagtctcccttccagtagtctgcttcagggtcctgagttctcttcctggcacgttttctgggtatagcaagagctggaactaccttgcataagtatgcaaggtagttccagaccttgccattcccaagtgccctggcagtcagtaggttgtgacaggctgagcagagagcttcttgggcttgcagcatctctcctgtcctccttgtcaggctccagagctgggggtgcccggactagtacatcatctatactgtagtgtctcatcgcaaacttacagtatatgatgatatctagatgcattcgcgaggtaccgagctcgaattcgcccttaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaggtaccgaattaagggcgaattcactggccgtcgttttacaacgtcgtgactgggaaaaccctggcgttacccaacttaatcgccttgcagcacatccccctttcgccagctggcgtaatagcgaagaggcccgcaccggatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccc

SEQ ID NO: 18 - protein, 621 aa, synthetic construct
  REGION 1..621: note = Rep78
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 19 - DNA, 1876 bp, synthetic construct
  misc_feature 1..1876: note = Rep78
  source 1..1876: mol_type = other DNA
cgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 20 - protein, 397 aa, synthetic construct
  REGION 1..397: note = Rep52
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 21 - DNA, 1194 bp, synthetic construct
  misc_feature 1..1194: note = Rep 52 wildtype
  source 1..1194: mol_type = other DNA
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagaccgctaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 22 - DNA, 1194 bp, synthetic construct
  misc_feature 1..1194: note = rep52 sf9 (insect cell) optimised
  source 1..1194: mol_type = other DNA
atggagctggtgggttggctggtggacaagggtatcacctccgagaagcagtggatccaggaggaccaggcttcctacatctccttcaacgctgcttccaactcccgttcccagatcaaggctgctctggacaacgctggtaagatcatgtccctgaccaagaccgctcctgactacctggtgggtcagcagcctgtggaggacatctcctccaaccgtatctacaagatcctggagctgaacggttacgaccctcagtacgctgcttccgtgttcctgggttgggctaccaagaagttcggtaagcgtaacaccatctggctgttcggtcctgctaccaccggtaagaccaacatcgctgaggctatcgctcacaccgtgcctttctacggttgcgtgaactggaccaacgagaacttccctttcaacgactgcgtggacaagatggtgatctggtgggaggagggtaagatgaccgctaaggtggtggagtccgctaaggctatcctgggtggttccaaggtgcgtgtggaccagaagtgcaagtcctccgctcagatcgaccctacccctgtgatcgtgacctccaacaccaacatgtgcgctgtgatcgacggtaactccaccaccttcgagcaccagcagcctctgcaggaccgtatgttcaagttcgagctgacccgtcgtctggaccacgacttcggtaaggtgaccaagcaggaggtgaaggacttcttccgttgggctaaggaccacgtggtggaggtggagcacgagttctacgtgaagaagggtggtgctaagaagcgtcctgctccttccgacgctgacatctccgagcctaagcgtgtgcgtgagtccgtggctcagccttccacctccgacgctgaggcttccatcaactacgctgaccgttaccagaacaagtgctcccgtcacgtgggtatgaacctgatgctgttcccttgccgtcagtgcgagcgtatgaaccagaactccaacatctgcttcacccacggtcagaaggactgcctggagtgcttccctgtgtccgagtcccagcctgtgtccgtggtgaagaaggcttaccagaagctgtgctacatccaccacatcatgggtaaggtgcctgacgcttgcaccgcttgcgacctggtgaacgtggacctggacgactgcatcttcgagcagtaa

SEQ ID NO: 23 - DNA, 1194 bp, synthetic construct
  misc_feature 1..1194: note = rep52 AT optimised
  source 1..1194: mol_type = other DNA
atggaattagtaggatggttagtagataaaggaataacatcagaaaaacaatggatacaagaagatcaagcatcatatatatcatttaatgcagcatcaaattcaagatcacaaataaaagcagcattagataatgcaggaaaaataatgtcattaacaaaaacagcaccagattatttagtaggacaacaaccagtagaagatatatcatcaaatagaatatataaaatattagaattaaatggatatgatccacaatatgcagcatcagtatttttaggatgggcaacaaaaaaatttggaaaaagaaatacaatatggttatttggaccagcaacaacaggaaaaacaaatatagcagaagcaatagcacatacagtaccattttatggatgtgtaaattggacaaatgaaaattttccatttaatgattgtgtagataaaatggtaatatggtgggaagaaggaaaaatgacagcaaaagtagtagaatcagcaaaagcaatattaggaggatcaaaagtaagagtagatcaaaaatgtaaatcatcagcacaaatagatccaacaccagtaatagtaacatcaaatacaaatatgtgtgcagtaatagatggaaattcaacaacatttgaacatcaacaaccattacaagatagaatgtttaaatttgaattaacaagaagattagatcatgattttggaaaagtaacaaaacaagaagtaaaagatttttttagatgggcaaaagatcatgtagtagaagtagaacatgaattttatgtaaaaaaaggaggagcaaaaaaaagaccagcaccatcagatgcagatatatcagaaccaaaaagagtaagagaatcagtagcacaaccatcaacatcagatgcagaagcatcaataaattatgcagatagatatcaaaataaatgttcaagacatgtaggaatgaatttaatgttatttccatgtagacaatgtgaaagaatgaatcaaaattcaaatatatgttttacacatggacaaaaagattgtttagaatgttttccagtatcagaatcacaaccagtatcagtagtaaaaaaagcatatcaaaaattatgttatatacatcatataatgggaaaagtaccagatgcatgtacagcatgtgatttagtaaatgtagatttagatgattgtatatttgaacaataa

SEQ ID NO: 24 - DNA, 1194 bp, synthetic construct
  misc_feature 1..1194: note = rep52 GC optimised
  source 1..1194: mol_type = other DNA
atggagctggtggggtggctggtggacaaggggatcacgagcgagaagcagtggatccaggaggaccaggcgagctacatcagcttcaacgcggcgagcaacagccggagccagatcaaggcggcgctggacaacgcggggaagatcatgagcctgacgaagacggcgccggactacctggtggggcagcagccggtggaggacatcagcagcaaccggatctacaagatcctggagctgaacgggtacgacccgcagtacgcggcgagcgtgttcctggggtgggcgacgaagaagttcgggaagcggaacacgatctggctgttcgggccggcgacgacggggaagacgaacatcgcggaggcgatcgcgcacacggtgccgttctacgggtgcgtgaactggacgaacgagaacttcccgttcaacgactgcgtggacaagatggtgatctggtgggaggaggggaagatgacggcgaaggtggtggagagcgcgaaggcgatcctgggggggagcaaggtgcgggtggaccagaagtgcaagagcagcgcgcagatcgacccgacgccggtgatcgtgacgagcaacacgaacatgtgcgcggtgatcgacgggaacagcacgacgttcgagcaccagcagccgctgcaggaccggatgttcaagttcgagctgacgcggcggctggaccacgacttcgggaaggtgacgaagcaggaggtgaaggacttcttccggtgggcgaaggaccacgtggtggaggtggagcacgagttctacgtgaagaaggggggggcgaagaagcggccggcgccgagcgacgcggacatcagcgagccgaagcgggtgcgggagagcgtggcgcagccgagcacgagcgacgcggaggcgagcatcaactacgcggaccggtaccagaacaagtgcagccggcacgtggggatgaacctgatgctgttcccgtgccggcagtgcgagcggatgaaccagaacagcaacatctgcttcacgcacgggcagaaggactgcctggagtgcttcccggtgagcgagagccagccggtgagcgtggtgaagaaggcgtaccagaagctgtgctacatccaccacatcatggggaaggtgccggacgcgtgcacggcgtgcgacctggtgaacgtggacctggacgactgcatcttcgagcagtaa

SEQ ID NO: 25 - DNA, 1194 bp, synthetic construct; identical to SEQ ID NO: 21
  misc_feature 1..1194: note = rep52
  source 1..1194: mol_type = other DNA

SEQ ID NO: 26 - protein, 724 aa, synthetic construct
  REGION 1..724: note = AAV5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 27 - DNA, 2211 bp, synthetic construct
  misc_feature 1..2211: note = AAV1, VP1, VP2, VP3 startcodon VP1 altered (GTG)
  source 1..2211: mol_type = other DNA
gtggctgccgacggttatctacccgattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 28 - DNA, 1800 bp, synthetic construct
  misc_feature 1..1800: note = AAV1, VP2, VP3
  source 1..1800: mol_type = other DNA
acggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 29 - DNA, 1605 bp, synthetic construct
  misc_feature 1..1605: note = AAV1, VP3
  source 1..1605: mol_type = other DNA
atggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 30 - DNA, 12538 bp, synthetic construct
  misc_feature 1..12538: note = Bac Trans
  source 1..12538: mol_type = other DNA
cgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccaggctgcagggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgataatacgactcactatagggagacccaagcttgatcccccctcttcctcctcctcaagggaaagctgcccacttctagctgccctgccatcccctttaaagggcgacttgctcagcgccaaaccgcggctccagccctctccagcctccggctcagccggctcatcagtcggtcaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttgtccgtgttgcttggtcttcacctgtgcagaattgcgaaccatggattcatcgacggtaccgcgggccctcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccccctgcagcctgcattaatgaatcggccaacgcgcggggagaggcggtttgcgtattgggcgctcttccgcttcctcgctcactgactcgctgcgctcggtcgttcggctgcggcgaggggggtaccagatcccatgggagctctgcagaattctctagaggcctcgcgagatcttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggccgacacccatttgaaaaaagcgaagacgaaatgttggacttgaatagcatgaagcgtcgtcagcaatacaatgacattggcgttttaaaacacgttcgtaacgttaacgctcgtgactttgtgtactgcctaacaagatacaacatagattgtagactcacaaattacaaacaaattataaaacatgagtttttgtcgtaaaaatgccacttgttttacgagtagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgat

SEQ ID NO: 31 - DNA, 11544 bp, synthetic construct
  misc_feature 1..11544: note = Bac PolH Cap2
  source 1..11544: mol_type = other DNA
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccatcatggagatctgagctcggcgcgtgtaatgagacgcacaaactaatatcacaaactggaaatgtctatcaatatatagttgctgatgtaccgcatgctatgcatcagctgctagtactccggaatattaatagatcatggagataattaaaatgataaccatctcgcaaataaataagtattttactgttttcgtaacagttttgtaataaaaaaacctataaatagaccggagtagtcataccgtcccaccatcgggcgcggatcgtaccgggcccaagcttgccgccaccctggctgccgatggttatctacccgattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggccgccccgggggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgtttttta

SEQ ID NO: 32 - DNA, 14299 bp, synthetic construct
  misc_feature 1..14299: note = Bac polH Cap5 - human Factor IX
  source 1..14299: mol_type = other DNA
tgtaatgagacgcacaaactaatatcacaaactggaaatgtctatcaatatatagttgctgatgtaccgcagcatgctatgcatcagctgctagtactccggaatattaatagatcatggagataattaaaatgataaccatctcgcaaataaataagtattttactgttttcgtaacagttttgtaataaaaaaacctataaatagaccggagtagtcataccgtcccaccatcgggcgcggatcgtaccgggcccaagcttcctgttaagacggcttcttttgttgatcacccacccgattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaatctagagcctgcagtctcgacaagctagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggggggtaccagatcccatgggagctctgcagaattctctagaggcctcgcgagatcgatctagaaagcttcccggggggatctgggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctggaggggtggagtcgtgacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgaattctagaccaccatgcagagggtgaacatgatcatggctgagagccctggcctgatcaccatctgcctgctgggctacctgctgtctgctgagtgcactgtgttcctggaccatgagaatgccaacaagatcctgaacaggcccaagagatacaactctggcaagctggaggagtttgtgcagggcaacctggagagggagtgcatggaggagaagtgcagctttgaggaggccagggaggtgtttgagaacactgagaggaccactgagttctggaagcagtatgtggatggggaccagtgtgagagcaacccctgcctgaatgggggcagctgcaaggatgacatcaacagctatgagtgctggtgcccctttggctttgagggcaagaactgtgagctggatgtgacctgcaacatcaagaatggcagatgtgagcagttctgcaagaactctgctgacaacaaggtggtgtgcagctgcactgagggctacaggctggctgagaaccagaagagctgtgagcctgctgtgccattcccatgtggcagagtgtctgtgagccagaccagcaagctgaccagggctgaggctgtgttccctgatgtggactatgtgaacagcactgaggctgaaaccatcctggacaacatcacccagagcacccagagcttcaatgacttcaccagggtggtggggggggaggatgccaagcctggccagttcccctggcaagtggtgctgaatggcaaggtggatgccttctgtgggggcagcattgtgaatgagaagtggattgtgactgctgcccactgtgtggagactggggtgaagatcactgtggtggctggggagcacaacattgaggagactgagcacactgagcagaagaggaatgtgatcaggatcatcccccaccacaactacaatgctgccatcaacaagtacaaccatgacattgccctgctggagctggatgagcccctggtgctgaacagctatgtgacccccatctgcattgctgacaaggagtacaccaacatcttcctgaagtttggctctggctatgtgtctggctggggcagggtgttccacaagggcaggtctgccctggtgctgcagtacctgagggtgcccctggtggacagggccacctgcctgctgagcaccaagttcaccatctacaacaacatgttctgtgctggcttccatgaggggggcagggacagctgccagggggactctgggggcccccatgtgactgaggtggagggcaccagcttcctgactggcatcatcagctggggggaggagtgtgccatgaagggcaagtatggcatctacaccaaagtctccagatatgtgaactggatcaaggagaagaccaagctgacctgactcgatgctttatttgtgaaatttgtgatgctattgctttatttgtaaccattataagctgcaataaacaagttaacaacaacaattgcattcattttatgtttcaggttcagggggaggtgtgggaggttttttaaactaggtcacgactccacccctccaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccagatccccccgggaagctttctagatcgatcttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggccgacacccatttgaaaaaagcgaagacgaaatgttggacttgaatagcatgaagcgtcgtcagcaatacaatgacattggcgttttaaaacacgttcgtaacgttaacgctcgtgactttgtgtactgcctaacaagatacaacatagattgtagactcacaaattacaaacaaattataaaacatgagtttttgtcgtaaaaatgccacttgttttacgagtagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccat

SEQ ID NO: 33 - DNA, 13365 bp, synthetic construct
  misc_feature 1..13365: note = Bac Rep183
  source 1..13365: mol_type = other DNA
accgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagtcgagattaccctgttatccctaccagtgtgttggatttattgttcaaagatacagtcatccaaatccacattaaccagatcgcaggcagtacaagcgtctggcacttttcccatgatatgatgaatatagcataatttttgatacgccttttttacgacagaaacgggttgagattctgacaccggaaagcattctaaacagtctttctggccgtgagtgaaacagatattactattctgattcattctctcacattgtctgcagggaaacaacattaagttcatgcctacgtgacgagaacatttgttttggtagcggtctgcgtagtttatcgaagcttccgcatctgacgtgcttggctgcgcaaccgattctctcactcgtttgggctcacttatatctgcatcactcggggcgggtcttttcttagcaccaccttttttgacgtaaaattcatgttccacttcaacaacgtgatcctttgcccaacgaaagaagtctttgacttcttgttttgttaccttgccaaaatcatgatccagtcggcgcgtcaattcaaatttgaacattcggtcttgcaacggttgttggtgttcgaatgtcgtactgttaccgtcaatcacggcgcacatgttcgtgttgcttgtaacgatcaccggtgtcgggtctatctgcgcagagcttttgcatttctggtctacgcgcactttgctgcctcctaaaattgctttggccgactccacgactttagcggtcattttgccttcctcccaccaaataaccatcttgtcgacacagtcgttgaatggaaagttctcattggtccagttaacgcagccataaaaaggtacagtgtgggctatggcctccgctatgtttgtttttcccgtagttgcaggtccaaacaaccaaatggtgtttcttttgccaaactttttcgtcgcccagcccaaaaatacggaagccgcatattgaggatcgtagccgtttaactccaaaatcttatagatgcgattgctggaaatgtcttccacgggttgctggcccaccaggtagtcgggggcggttttagtcaggctcataatcttgcccgcattgtccaaggcagctttgatttggctacgcgagttggatgccgcattaaacgagatgtatgaggcttgatcttcttgtatccattgcttctccgaggtaatacccttgtccaccaaccaaccgaccaattccatggcgaccgagatccgcgcccgatggtgggacggtatgaataatccggaatatttataggtttttttattacaaaactgttacgaaaacagtaaaatacttatttatttgcgagatggttatcattttaattatctccatgatctattaatattccggagtactgctagcaccatggatcccggtccgaagcgcgcggaattcaaaggcctacgtcgacgagctcactagtcgcggccgatctaataaacgataacgccgggtggcgtgaggcatgtaaaaggttacatcattatcttgttcgccatccggttggtataaatagacgttcatgttggtttttgtttcagttgcaagttggctgcggcgcgcgcagcacctttgcggccatctgcagaattcgcccttgttactcttcagccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaggcctcgcgagatcttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggccgacacccatttgaaaaaagcgaagacgaaatgttggacttgaatagcatgaagcgtcgtcagcaatacaatgacattggcgttttaaaacacgttcgtaacgttaacgctcgtgactttgtgtactgcctaacaagatacaacatagattgtagactcacaaattacaaacaaattataaaacatgagtttttgtcgtaaaaatgccacttgttttacgagtagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagt

SEQ ID NO: 34 - DNA, 250 bp, synthetic construct
  misc_feature 1..250: note = PolH short
  source 1..250: mol_type = other DNA
tgtaatgagacgcacaaactaatatcacaaactggaaatgtctatcaatatatagttgctgatctatgcatcagctgctagtactccggaatattaatagatcatggagataattaaaatgataaccatctcgcaaataaataagtattttactgttttcgtaacagttttgtaataaaaaaacctataaatattccggattattcataccgtcccaccatcgggcgcggatcgtaccgggcccaagctt

SEQ ID NO: 35 - DNA, 155 bp, synthetic construct
  misc_feature 1..155: note = PolH long
  source 1..155: mol_type = other DNA
tgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaat

SEQ ID NO: 36 - DNA, 28 bp, synthetic construct
  misc_feature 1..28: note = Hr 28-mer
  source 1..28: mol_type = other DNA
ctttacgagtagaattctacgcgtaaaa

SEQ ID NO: 37 - DNA, 7311 bp, synthetic construct
  misc_feature 1..7311: note = pCLD002
  source 1..7311: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcataccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgagggcccgcggtaccgtcgatgaatccatggttcgcaattctgcacaggtgaagaccaagcaacacggacttaaccctcccaaacataaccagaggggagaagttcacgaataccgacggcggttggctggccgtctgaatcttttacaatagctttaatgcctggatgaaggtcaagaagtacctgacgacacctgccgcatggggaaagaataccacggttttcatttccaatcgccacgatacacgtaaggttacccgctgctgccgcagccgcagtacccaatacgaccagctccgcacaagggcctccggtgaaatggtacacgttaacacccgtgaagatgcgtccatccgacgagagcgccgcacttgcaactgagtaatcttccgaaattggtatagaattgatcgtggcggtagcacgctcaatcaatgtagattcctcctgtgagaggggttttgccatggtggcgaccggtagcctcgagtaccggatcctctagcggccgaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtataaaggtgcttgttggcaaacatgaaaacacagttcaacagaattgttgttgaagcaacattagcaccatacattgtttatcatcatgaataacttcgtataatgtatgctatacgaagttatttgcggccgcttgatatcttcctgcaggttatcgatttggccgcgaattcactagtgattgcggaataattgccatatgtaaatgatgtcatcgttctaactcgctttacgagtagaattctacgtgtaaaacataatcaagagatgatgtcatttgtttttcaaaactgaactcaagaaatgatgtcatttgtttttcaaaactgaactggctttacgagcagaattctacttgtaacgcatgatcaagggatgatgtcatttgttttttaaaattgaactggctttacgagtagaattctacttgtaaaacacaatcgagagatgatgtcatattttgcacacggctctaattaaactcgctttacgagtaaaattctacttgtaacgcatgatcaagggatgatgtcattggatgagtcatttgtttttcaaaactaaactcgctttacgagtagaattctacttgtaaaacacaatcaagggatgatgtcattatacaaatgatgtcatttgtttttcaaaactaaactcgctttacgggtagaattctacttgtaaaacacaatcgagggatgatgtcatcctttacacatgattataaacgtgtttatgtatgactcatttgtttttcaaaactaaactcgctttacgagtagaattctacttgtaacgcacgatcaagggatgatgtcatttatttgtgcaaagctgatgtcatcttttgcacacgattataaacacaatcaaataatgactcatttgttttcaaaactgaactcgctttacgagtagaattctacttgtaaaacacaatcaagggatgatgtcattttaaaaatgatgtcatttgtttttcaaaactaaactcgctttacgagtagaattctacgtgtaaaacacaatcaagggatgatgtcatttactaaaataaaataattatttaaataaaaatgtttttattgtaaaatacacattgattacacgtgacaatcgaattcccgcttgctagcttcttaagttagatctttatgcatttcggagcgagaccatcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcaaaggcctacgtcgacgagctcactagtaacggccgccagtgtgctggaattcgcccttcgcggatcctgttaagacggcggggttctacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggtctcaccatgcgtacagcttgacgcgtgcgtaataacttcgtataatgtatgctatacgaagttatactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 38 - DNA, 2159 bp, synthetic construct
  misc_feature 1..2159: note = BacTrans6
  source 1..2159: mol_type = other DNA
gatccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctaagcaaatatttgtggttatggattaactcgaactgtttgcccactctatttgcccggcgccctttggaccttttgcaatcctggagcaaacagcaaacacggacttagcccctgtttgctcctccgataactggggtgaccttggttaatattcaccagcagcctcgggcatataaaacaggggcaaggcacagactcatagcagagcaatcaccaccaagcctggaataactgcagccaccatgcagagggtgaacatgatcatggctgagagccctggcctgatcaccatctgcctgctgggctacctgctgtctgctgagtgcactgtgttcctggaccatgagaatgccaacaagatcctgaacaggcccaagagatacaactctggcaagttcgaggagtttgtgcagggcaacctggagagggagtgcatggaggagaagtgcagctttgaggaggccagggaggtgtttgagaacactgagaggaccactgagttctggaagcagtatgtggatggggaccagtgtgagagcaacccctgcctgaatgggggcagctgcaaggatgacatcaacagctatgagtgctggtgcccctttggctttgagggcaagaactgtgagctggatgtgacctgcaacatcaagaatggcagatgtgagcagttctgcaagaactctgctgacaacaaggtggtgtgcagctgcactgagggctacaggctggctgagaaccagaagagctgtgagcctgctgtgccattcccatgtggcagagtgtctgtgagccagaccagcaagctgaccagggctgaggctgtgttccctgatgtggactatgtgaacagcactgaggctgaaaccatcctggacaacatcacccagagcacccagagcttcaatgacttcaccaggatcgtggggggggaggatgccaagcctggccagttcccctggcaagtggtgctgaatggcaaggtggatgccttctgtgggggcagcattgtgaatgagaagtggattgtgactgctgcccactgtgtggagactggggtgaagatcactgtggtggctggggagcacaacattgaggagactgagcacactgagcagaagaggaatgtgatcaggatcatcccccaccacaactacaatgctgccatcaacgcctacaaccatgacattgccctgctggagctggatgagcccctggtgctgaacagctatgtgacccccatctgcattgctgacaaggagtacaccaacatcttcctgaagtttggctctggctatgtgtctggctggggcagggtgttccacaagggcaggtctgccctggtgctgcagtacctgagggtgcccctggtggacagggccacctgcctgaggagcaccaagttcaccatctacaacaacatgttctgtgctggcttccatgaggggggcagggacagctgccagggggactctgggggcccccatgtgactgaggtggagggcaccagcttcctgactggcatcgtgagctggggggaggagtgtgccatgaagggcaagtatggcatctacaccaaagtctccagatatgtgaactggatcaaggagaagaccaagctgacctgactcgatgctttatttgtgaaatttgtgatgctattgctttatttgtaaccattataagctgcaataaacaagttaacaacaacaattgcattcattttatgtttcaggttcagggggaggtgtgggaggttttttaaaagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

(19) United States
(12) Patent Application Publication
(10) Pub. No.: US 2023/0159951 A1
(43) Pub. Date: May 25, 2023

(54) DUAL BIFUNCTIONAL VECTORS FOR AAV PRODUCTION

(71) Applicant: uniQure biopharma B.V., Amsterdam (NL)

(72) Inventors: David Johannes Francois DU PLESSIS, Amsterdam (NL); ANGGAKUSUMA, Amsterdam (NL); Sebastiaan Menno BOSMA, Amsterdam (NL); Jacek LUBELSKI, Amsterdam (NL)

(21) Appl. No.: 17/948,868

(22) Filed: Sep. 20, 2022

Related U.S. Application Data
(63) Continuation of application No. PCT/EP2021/058794, filed on Apr. 2, 2021.

(30) Foreign Application Priority Data
Apr. 2, 2020 (EP) 20167813.3

(51) Int. Cl. C12N 15/86

(52) U.S. Cl. CPC C12N 15/86; C12N 2710/14144; C12N 2750/14151

(57) ABSTRACT

The present invention relates novel combinations of nucleic acid constructs for the production of recombinant parvoviral gene therapy vectors. In particular the invention relates a combination preferably no more than two construct, the first construct expressing both the parvoviral Cap and Rep proteins, and the second construct at least comprising the transgene flanked ITRs and optionally again comprising an expression cassette forthe Cap proteins. The nucleic acid constructs are preferably baculoviral vectors for the production of rAAV in insect cells.

### CROSS-REFERENCE TO RELATED APPLICATIONS

The present application is a continuation application of PCT/EP2021/058794 filed Apr. 2, 2021, which claims priority to EP 20167813.3 filed Apr. 2, 2020, the entire contents of both which are incorporated herein by reference.

### REFERENCE TO SEQUENCE LISTING

The present application is filed along with a Sequence Listing in electronic format. The Sequence Listing is provided as a file entitled 069818-0935.xml, created on Jan. 8, 2023, which is 199,973 bytes in size. The information in the electronic format of the Sequence Listing is incorporated herein by reference in its entirety.

### FIELD OF THE INVENTION

The present invention relates to the fields of medicine, molecular biology, and gene therapy. The invention relates to production of proteins in cells whereby repeated imperfect palindromic/homologous repeat sequences are used in baculoviral vectors. In particular, the invention relates to the production of parvoviral vectors that may be used in gene therapy, and, to improvements in expression of the viral replicase (Rep) proteins that increase the productivity of parvoviral vectors.

### BACKGROUND OF THE INVENTION

The baculovirus expression system is well known for its use as eukaryotic cloning and expression vector (King, L. A., and R. D. Possee, 1992, “The baculovirus expression system”, Chapman and Hall, United Kingdom; O’Reilly, D. R., et al., 1992. Baculovirus Expression Vectors: A Laboratory Manual. New York: W. H. Freeman). Advantages of the baculovirus expression system are, among others, that the expressed proteins are almost always soluble, correctly folded and biologically active. Further advantages include high protein expression levels, faster production, suitability for expression of large proteins and suitability for large-scale production. However, in large-scale or continuous production of heterologous proteins using the baculovirus system in insect cell bioreactors, the instability of production levels, also known as the passage effect, is a major obstacle. This effect is, at least in part, due to recombination between repeated homologous sequences in the baculoviral DNA.

The baculovirus expression system has also successfully been used for the production of recombinant adeno-associated virus (rAAV) vectors (Urabe et al., 2002, Hum. Gene Ther. 13: 1935-1943; US 6,723,551 and US 20040197895). AAV may be considered as one of the most promising viral vectors for human gene therapy. To date, two platforms have emerged as the main production systems capable of delivering research and clinical grade AAV material. In both cases, expression cassettes comprising replicase (Rep, DNA replication and packaging proteins) and capsid (Cap, structural proteins) encoding genes are delivered to the producer cell alongside a to-be-packaged transgene flanked by AAV2 inverted terminal repeats (ITRs). One approach relies on the transient chemical transfection of plasmids into Hek293 cells to deliver these components and produce AAV. In the second approach, Baculovirus expression vectors (BEVs) deliver the components to a suspension culture of invertebrate cells. While the mammalian cell-based production system for rAAV can produce high titer AAV material, it is less suitable for scale-up. This is mostly due to the high cost of plasmid production and the need to adapt Hek293 cells both to growth in suspension and AAV production, and even then yields are not in the same order as with insect cells. In contrast, the BEVs production system presents a more scalable platform for rAAV production because baculoviruses, once generated and characterized, can be amplified alongside insect cells, grown in suspension, prior to inoculation for AAV production. In general, yields on a per cell basis are comparable for suspension insect cells and adherent Hek293 cells.

The most frequently used method for producing rAAV in insect cells is via the co-infection of three separate baculoviruses, the TripleBac system. These baculoviruses comprise Rep, Cap and transgene (Trans) expression cassettes, respectively. The major drawback of using a co-infection of three baculoviruses during rAAV production is that non-simultaneous infection can occur. By creating baculoviral vectors each containing double expression cassettes, termed herein the DuoBac system (wherein each vector contains either Cap and Rep or Cap and Trans, FIG. 1), the number of different baculoviral vectors needed for rAAV production can be reduced thereby improving the chances for simultaneous infection. This reduction in process complexity has several potential benefits: 1. lower risk of contamination; 2. an average higher AAV yield in Crude Lysate Bulk (CLB); 3. a more robust baculovirus MOI response; 4. increased compatibility with upscaling; 5. lower Cost of Goods as one less seed virus is required; and 6. reduction of the total/full ratio of an AAV batch. All these advantages arise because the molecular components required for successful AAV production are more likely to be present in the cell at the right time.

For AAV productions that use baculoviruses in insect cells, optimizing the Cap and Rep protein expression in both time and amount is of critical importance for the quantity and quality of the produced AAVs. Previously, it was observed that early Rep78 expression (replicating Rep) and late Rep52 expression (packaging Rep) improved quality of the produced AAVs (US 6,697,417). Control over the timing of expression can be exercised by utilizing different baculovirus promoters that become active at different phases of infection (Chaabihi, H., et al., 1993, J Virol 67(5), 2664-71; Hill-Perkins, M. S. and Possee, R. D., 1990, J Gen Virol 71(4), 971-6; Pullen, S. S. and Friesen, P. D., 1995, J Virol 69(1), 156-65). The immediate early (IE) promoter is active at the early stage of baculovirus infection, immediately after infection, but declines thereafter. The p10 and polyhedrin promoters are both strong but very late promoters, where peak expression is observed at 20-24 hours after infection. By separating the Rep52 and Rep78 expression cassettes and controlling their expression with different promoters the current inventors have better control of the individual strength and timing of the Rep proteins and thereby improve quality of the produced AAVs. In addition, in application WO2007/148971 the present inventors have significantly improved the stability of rAAV vector production in insect cells by using a single coding sequence for the Rep78 and Rep52 proteins wherein a suboptimal initiator codon is used for the Rep78 protein that is partially skipped by the scanning ribosomes to allow for initiation of translation to also occur further downstream at the initiation codon of the Rep52 protein. In WO 2009/014445 the stability of rAAV vector production in insect cells was again further improved by employing separate expression cassettes for the Rep52 and Rep78, wherein the repeated coding sequences differ in codon bias to reduce homologous recombination.

Stoichiometry of the capsid proteins (VP1, VP2 and VP3) needs to be as close as possible to the natural ratio of 1:1:10. VP1 contains phospholipase A2 activity and is essential for endosomal escape once a capsid enters the cell. If this ratio falls outside its optimum the capsid will be less potent, for example, low VP1 generally leads to poor infectivity (as measured in cell entry and transgene expression) but a high titer AAV production (in gc/ml). The combination of the chosen capsid promoter and VP1 start codon together exert the biggest influence on this ratio and needs to be optimized for the individual AAV serotypes. Mixing different promoter strengths and VP1 start codons can alter the VP1:2:3 ratio of the produced capsids and thereby its potency (Bosma, B., et al., 2018, Gene Ther 25(6), 415-424). International patent application WO 2007/084773 discloses a method of rAAV production in insect cells, wherein the production of infectious viral particles are increased by supplementing VP1 relative to VP2 and VP3. Supplementation can be affected by introducing into the insect cell a capsid vector comprising nucleotide sequences expressing VP1, VP2 and VP3 and additionally introducing into the insect cell nucleotide sequences expressing VP1, which may be either on the same capsid vector or on a different vector.

In the past, baculovirus constructs containing double expression cassettes were designed around AAV serotype 1 (WO2009/104964). While these constructs displayed an improved total/full ratio and normal capsid stoichiometry, virus yields were approximately three fold lower than TripleBac AAV1 productions. One explanation for the reduced yields may be due to the use of a single Rep expression cassette, where timing of expression, as well as Rep52 and Rep78 ratio, was suboptimal. This likely led to high foreign (non-AAV) DNA encapsidation in the particle and low yields. Therefore, there is still a need for means and methods to improve the quality and quantity of recombinant parvoviral gene therapy vectors such as rAAV.

### SUMMARY OF THE INVENTION

In a first aspect the invention relates to a cell comprising one or more nucleic acid constructs comprising: i) a first expression cassette comprising a first promoter operably linked to a nucleotide sequence encoding an mRNA, translation of which in the cell produces at least one of parvoviral Rep 78 and 68 proteins; ii) a second expression cassette comprising a second promoter operably linked to a nucleotide sequence encoding an mRNA, translation of which in the cell produces at least one of parvoviral Rep 52 and 40 proteins; iii) a third expression cassette comprising a third promoter operably linked to a nucleotide sequence encoding parvoviral VP1, VP2, and VP3 capsid proteins; and, iv) a nucleotide sequence comprising a transgene that is flanked by at least one parvoviral inverted terminal repeat sequence, wherein, at least one of the first and second expression cassette are present on a first nucleic acid construct with the third expression cassette, and wherein, upon transfection of the cell with the one or more nucleic acid constructs, the first promoter is active before the second and third promoters. Preferably, the nucleotide sequence comprising the transgene flanked by the parvoviral inverted terminal repeat sequence is present on a second nucleic acid construct. Preferably, the second nucleic acid construct further comprises a fourth expression cassette comprising a fourth promoter operably linked to a nucleotide sequence encoding parvoviral VP1, VP2, and VP3 capsid proteins, wherein the first promoter is active before the second, third and fourth promoters, wherein optionally, the third and fourth promoters are identical, and wherein optionally, the parvoviral VP1, VP2, and VP3 capsid proteins encoded by the nucleotide sequences in the third and fourth expression cassettes are identical.

In a preferred embodiment, the at least one of parvoviral Rep 78 and 68 proteins and the at least one of parvoviral Rep 52 and 40 proteins comprise a common amino acid sequence comprising the amino acid sequence from the second amino acid to the most C-terminal amino acid of the at least one of parvoviral Rep 52 and 40 proteins, wherein the common amino acid sequences of the at least one of parvoviral Rep 78 and 68 proteins and the at least one of parvoviral Rep 52 and 40 proteins are at least 90% identical, and wherein the nucleotide sequence encoding the common amino acid sequence of the at least one of parvoviral Rep 78 and 68 proteins and the nucleotide sequence encoding the common amino acid sequences of the at least one of parvoviral Rep 52 and 40 proteins are less than 90% identical. Preferably, the common amino acid sequences of the at least one of parvoviral Rep 78 and 68 proteins and the at least one of parvoviral Rep 52 and 40 proteins are at least 99% identical, preferably 100% identical. It is further preferred that the nucleotide sequence encoding the common amino acid sequence of the at least one of parvoviral Rep 78 and 68 proteins has an improved codon usage bias for the cell as compared to the nucleotide sequence encoding the common amino acid sequences of the at least one of parvoviral Rep 52 and 40, or wherein the nucleotide sequence encoding the common amino acid sequence of the at least one of parvoviral Rep 52 and 40 proteins has an improved codon usage bias for the cell as compared to the nucleotide sequence encoding the common amino acid sequences of the at least one of parvoviral Rep 78 and 68 proteins, wherein more preferably, the difference in codon adaptation index between the nucleotide sequences coding for the common amino acid sequences in the at least one of parvoviral Rep 78 and 68 proteins and the at least one of parvoviral Rep 52 and 40 proteins is at least 0.2.

In one embodiment, the first promoter is a constitutive promoter.

In one embodiment, at least one of the second, third and fourth promoters is an inducible promoter. Preferably, the inducible promoter is a viral promoter that is induced at a later stage in the virus’ infection cycle, preferably the viral promoter that is induced at least 24 hours after transfection or infection of the cell with the virus.

In one embodiment, at least one of the first and second nucleic acid construct is stably integrated in the genome of the cell.

In a preferred embodiment, the cell is an insect cell, and wherein at least one the first and second nucleic acid construct is an insect cell-compatible vector, preferably a baculoviral vector. Preferably in the insect cell, a) the first promoter is selected from a deltaEI promoter and an EI promoter; and, b) the second, third and fourth promoters are selected from a poIH promoter and a p10 promoter. More preferably in the insect cell, at least one expression cassette comprises at least one baculovirus enhancer element and/or at least one ecdysone responsive element, wherein preferable the enhancer element is selected from the group consisting of hr1, hr2, hr2.09, hr3, hr4, hr4b and hr5, preferably selected from the group hr2.09, hr4b and hr5.

In one embodiment, the nucleotide sequence encoding an mRNA, translation of which in the cell produces only at least one of parvoviral Rep 78 and 68 proteins, comprises an intact parvoviral p19 promoter.

In a preferred embodiment, the at least one of parvoviral Rep 78 and 68 proteins, the at least one of parvoviral Rep 52 and 40 proteins, the parvoviral VP1, VP2, and VP3 capsid proteins and the at least one parvoviral inverted terminal repeat sequence are from an adeno associated virus (AAV).

In one embodiment, the first nucleic acid construct is DuoBac CapRep6 (SEQ ID NO. 10) and the second nucleic acid construct is DuoBac CapTrans1 (SEQ ID NO. 12), and wherein the first and second constructs are preferably present in a 3 : 1 molar ratio.

In a second aspect, the invention pertains to a method for producing a recombinant parvoviral virion in a cell comprising the steps of: a) culturing a cell as defined herein under conditions such that recombinant parvoviral virion is produced; and, b) recovery of the recombinant parvoviral virion. Preferably in the method, the cell is an insect cell and/or wherein the parvoviral virion is an AAV virion. In a preferred method, recovery of the recombinant parvoviral virion in step b) comprises at least one of affinity-purification of the virion using an immobilised anti-parvoviral antibody, preferably a single chain camelid antibody or a fragment thereof, or filtration over a filter having a nominal pore size of 30 - 70 nm.

In a third aspect the invention relates to a nucleic acid construct as defined herein, specifically to the first and second nucleic acid constructs as defined herein.

In a fourth aspect, the invention pertains to a kit of parts comprising at least a first and second nucleic acid construct as defined herein.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, ... ,etc.

The word “about” or “approximately” when used in association with a numerical value (e.g. about 10) preferably means that the value may be the given value (of 10) more or less 0.1% of the value. The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using local alignment algorithms (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty = 50 (nucleotides) / 8 (proteins) and gap extension penalty = 3 (nucleotides) / 2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall length, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403—10. BLAST nucleotide searches can be performed with the NBLAST program, score = 100, wordlength = 12 to obtain nucleotide sequences homologous to oxidoreductase nucleic acid molecules of the invention. BLAST protein searches can be performed with the BLASTx program, score = 50, wordlength = 3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6X sodium chloride/sodium citrate (SSC) at about 45° C., followed by one or more washes in 1 X SSC, 0.1% SDS at about 50° C., preferably at about 55° C., preferably at about 60° C. and even more preferably at about 65° C.

Highly stringent conditions include, for example, hybridization at about 68° C. in 5x SSC/5x Denhardt’s solution / 1.0% SDS and washing in 0.2x SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3^{rd} edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. A “vector” is a nucleic acid construct (typically DNA or RNA) that serves to transfer an exogenous nucleic acid sequence (i.e., DNA or RNA) into a host cell. The terms “expression vector” or “expression construct” refer to nucleotide sequences that are capable of affecting expression of a gene in host cells or host organisms compatible with such sequences. These expression vectors typically include at least one “expression cassette” that is the functional unit capable of affecting expression of a sequence encoding a product to be expressed and wherein the coding sequence is operably linked to the appropriate expression control sequences, which at least comprises a suitable transcription regulatory sequence and optionally, 3′ transcription termination signals. Additional factors necessary or helpful in affecting expression may also be present, such as expression enhancer elements. The expression vector will be introduced into a suitable host cell and be able to affect expression of the coding sequence in an in vitro cell culture of the host cell. The expression vector will be suitable for viral vector, particularly recombinant AAV vector, replication in the host cell or organism of the invention.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer or biological entity.

The term “reporter” may be used interchangeably with marker, although it is mainly used to refer to visible markers, such as green fluorescent protein (GFP) or luciferase.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO’s) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which they are introduced but have been obtained from another cell or are synthetically or recombinantly produced. Generally, though not necessarily, such nucleic acids encode proteins, i.e. exogenous proteins, that are not normally produced by the cell in which the DNA is transcribed or expressed. Similarly, exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

As used herein, the term “non-naturally occurring” when used in reference to an organism means that the organism has at least one genetic alternation that is not normally found in a naturally occurring strain of the referenced species, including wild-type strains of the referenced species. Genetic alterations include, for example, modifications introducing expressible nucleic acids encoding proteins or enzymes, other nucleic acid additions, nucleic acid deletions, nucleic acid substitutions, or other functional disruption of the organism’s genetic material. Such modifications include, for example, coding regions and functional fragments thereof for heterologous or homologous polypeptides for the referenced species. Additional modifications include, for example, non-coding regulatory regions in which the modifications alter expression of a gene or operon. Genetic modifications to nucleic acid molecules encoding enzymes, or functional fragments thereof, can confer a biochemical reaction capability or a metabolic pathway capability to the non-naturally occurring organism that is altered from its naturally occurring state.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

“Expression cassette” refers to a nucleic acid sequence comprising an expression control sequence and a nucleic acid sequence to be expressed.

“Expression control sequence” or “regulatory control sequence” refers to a nucleic acid sequence that regulates the expression of a nucleotide sequence to which it is operably linked.

An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons.

The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e. a string of adenine residues at the 3′-end of a mRNA, sequences referred to as polyA sequences. It also can be designed to enhance mRNA stability. Expression control sequences which affect the transcription and translation stability, e.g., promoters, as well as sequences which affect the translation, e.g., Kozak sequences, are known in insect cells. Expression control sequences can be of such nature as to modulate the nucleotide sequence to which it is operably linked such that lower expression levels or higher expression levels are achieved.

The term “full virion” refers to a virion particle that comprises parvoviral structural/capsid proteins (VP1:2:3) encapsulating the transgene DNA flanked by inverted terminal repeat (ITR) sequences. The term “empty virion” refers to a virion particle that does not comprise the parvoviral genomic material. In a preferred embodiment of the invention, the full virion versus empty virion ratio is at least 1:50, more preferably at least 1:10, and even more preferably at least 1:1. Even more preferably, no empty virions can be detected and most preferably no empty virions are present. The person skilled in the art will know how to determine the full virion versus empty virion ratio, for example by dividing gene copy number by total particle with assembled AAV capsid number (or total assembled capsid:genome copy number), since per virion there will be only one genome copy present. The skilled artisan will know how to determine such ratios. For example, the ratio of empty virions versus total capsids may be determined by dividing the amount of genome copies (i.e. genome copy number) by the amount of total parvoviral particles (i.e. number of parvoviral particles), wherein the amount of genome copies per ml is measured by quantitative PCR and the amount of total parvoviral particles per ml is measured with an enzyme immunoassay, e.g. from Progen.

The term “TripleBac” as used herein refers to a system of baculoviral vectors for producing rAAV in insect cells that require co-infection of three separate baculoviral vectors, i.e. three distinct baculoviral vectors for respectively each of the Rep, Cap and Trans expression cassettes. The term “DuoBac” as used herein, refers to a system which uses only two different baculoviral vectors, one of which comprises two expression cassettes, for example, comprising Cap and Rep expression cassettes or comprising Cap and Trans cassettes. The term “DuoDuoBac” as used herein, refers to the system which uses two distinct baculoviral vectors each of which comprises at least two different expression cassettes, for example, one vector comprises the Cap and Rep cassettes and the other vector comprises the Cap and Trans cassettes.

### DETAILED DESCRIPTION OF THE INVENTION

The expression kinetics and ratio among parvoviral, i.e. AAV, structural and non-structural proteins, are important for the yield and quality of vector output from a production platform, especially using the baculovirus and insect cell platform. The vector quality is strongly related with the ratio between full virion versus empty virion, which contributes to potency of the vector itself.

The current inventors have further optimised production of rAAV in insect cells from baculoviral vectors amongst others by one or more of 1) using two DuoBac vectors, i.e. a Cap-Rep baculoviral vector and a Cap-Trans baculoviral vector (referred to as “DuoDuoBac” AAV production, see FIG. 1), 2) optimizing the promoter/VP1 start codon combination and 3) swapping the single Rep expression cassette for a double Rep expression cassette. The advantage of using the DuoDuoBac system, wherein a Cap-Rep baculoviral vector is combined with a Cap-Trans baculoviral vector, is that more control over the Cap:Rep ratio during AAV production is achieved. Previous TripleBac AAV production experiments showed that changing the Cap:Rep baculovirus inoculation ratio had an impact on the total/full ratio and AAV yield (in gc/ml).

The current inventors have found that increasing the amount of Rep during rAAV production represses both the capsid formation and total/full ratio, while increasing the amount of Cap increases the total/full ratio as well as the yield. As above, it would be known to one skilled in the art that the total/full ratio is one parameter that can be used to characterize an AAV batch. The total/full ratio, as used herein, refers to the ratio of DNA filled AAV particles (expressed in gc/ml) over the total number of AAV particles (expressed in VP/ml). Consequently, a lower total/full ratio means less empty particles per full particle and vice versa. Reducing the total/full ratio of produced AAV can potentially be beneficial for an AAV product because less particles can be dosed to achieve a similar amount of genome copies per kilogram. A low total/full ratio also results in a more homogenous product profile which is beneficial for setting up a robust downstream processes.

In addition, because the number of baculoviruses for inoculation is reduced, higher Cap:Rep ratios can be explored, which normally cannot be inoculated in a TripleBac system. In the TripleBac system, the reduction in the number of inoculated baculoviruses means that the overall baculovirus volume that gets added to a production culture is also lower. It is known in the art that adding high inoculation volumes to an AAV production was undesirable. Firstly, because large volumes of baculovirus are difficult to produce robustly and secondly, because the addition of a large volume of baculovirus to an AAV production inhibits the production. This is believed inter alia to be because of the addition of a large volume of spent media to a production culture.

In a first aspect, the invention therefore provides a cell comprising one or more nucleic acid constructs comprising: i) a first expression cassette comprising a first promoter operably linked to a nucleotide sequence encoding an mRNA, translation of which in the cell produces at least one of parvoviral Rep 78 and 68 proteins; ii) a second expression cassette comprising a second promoter operably linked to a nucleotide sequence encoding an mRNA, translation of which in the cell produces at least one of parvoviral Rep 52 and 40 proteins; iii) a third expression cassette comprising a third promoter operably linked to a nucleotide sequence encoding parvoviral VP1, VP2, and VP3 capsid proteins; and, iv) a nucleotide sequence comprising a transgene that is flanked by at least one parvoviral inverted terminal repeat sequence, wherein, at least one of the first and second expression cassette are present on a first nucleic acid construct with the third expression cassette, and wherein, upon transfection of the cell with the one or more nucleic acid constructs, the first promoter is active before the second and third promoters. The cell is preferably an insect cell as e.g. herein defined below. The nucleotide sequences encoding the mRNAs, translation of which produces either at least one of parvoviral Rep 52 and 40 proteins or at least one of parvoviral Rep 78 and 68 proteins preferably are nucleotide sequences as described herein below. The nucleotide sequence encoding the parvoviral VP1, VP2, and VP3 capsid proteins preferably is a nucleotide sequence as described herein below. The nucleotide sequence comprising the transgene flanked by one or more parvoviral inverted terminal repeats is described in further detail below. The first nucleic acid construct is thus preferably a single type of nucleic acid construct comprising each of the first, second and third expression cassettes. In one embodiment, the first nucleic acid construct does not comprise transgene flanked by one or more parvoviral inverted terminal repeats.

In one embodiment therefore, the nucleotide sequence comprising the transgene flanked by the parvoviral inverted terminal repeat sequence is present on a second nucleic acid construct. The second nucleic acid construct preferably is different from the first nucleic acid construct.

In a preferred embodiment, the second nucleic acid construct further comprises a fourth expression cassette comprising a fourth promoter operably linked to a nucleotide sequence encoding parvoviral VP1, VP2, and VP3 capsid proteins, wherein the first promoter is active before the second, third and fourth promoters. Preferably, the parvoviral VP1, VP2, and VP3 capsid proteins encoded by the nucleotide sequences in the third and fourth expression cassettes are identical. The third and fourth promoters can be identical or they can be different promoters.

Suitable promoters to be applied as first, second, third and/or fourth promoters in the constructs of the invention are described in more details below.

### Replicase Proteins

Parvoviral, especially AAV, replicases, i.e. Rep proteins, are non-structural proteins encoded by the rep gene cassette. Due to endogenous P19 promoter, the gene produces two overlapping messenger ribonucleic acids (mRNA) with different length. Each of these mRNA can be spliced out or not to eventually generate four Rep proteins, Rep78, Rep68, Rep52 and Rep40. The Rep78/68 and Rep52/40 are important for the ITR-dependent AAV genome or transgene replication and viral particle assembly. Rep78/68 serves as a viral replication initiator proteins and act as replicase for the viral genome (Chejanovsky, N., Carter, B. J.. Mutation of a consensus purine nucleotide consensus binding site in the adeno-associated virus rep gene generates a dominant negative phenotype for DNA replication, J Virol., 1990, 64:1764-1770, Hong, G., Ward, P., Berns, K. I., In vitro replication of adeno-associated virus DNA, Proc Natl Acad Sci USA, 1992, 89:4673-4677. Ni. T-H., et al., In vitro replication of adeno-associated virus DNA, J Virol., 1994, 68:1128-1138). The Rep52/40 protein is DNA helicase with 3′ to 5′ polarity and plays a critical role during packaging of viral DNA into empty capsids, where they are thought to be part of the packaging motor complex (The Rep52 Gene Product of Adeno-Associated Virus Is a DNA Helicase with 3′-to-5′ Polarity; Smith and Kotin, J. Virol., 1998, 4874 - 4881, DNA helicase-mediated packaging of adeno-associated virus type 2 genomes into preformed capsids. King, J. A., et al., EMBO J., 2001, 20:3282-3291). To produce AAV from the baculovirus and insect cell platform, the present of both Rep68 and Rep40 is not prerequisite (Urabe, et al., 2002).

According to the invention, the cell comprises a first nucleic acid construct that comprises at least a first and a second expression cassette for expression of the parvoviral Rep proteins. The first expression cassette comprises a first promoter operably linked to a nucleotide sequence encoding an mRNA, translation of which in the cell produces at least one of parvoviral Rep 78 and 68 proteins.

In a preferred embodiment, the first expression cassette comprises a first promoter operably linked to a nucleotide sequence encoding an mRNA, translation of which in the cell produces only at least one of parvoviral Rep 78 and 68 proteins. Thereby it is understood that the nucleotide sequence encoding the parvoviral Rep 78 and/or 68 proteins encodes an open reading frame for the parvoviral Rep 78 and/or 68 proteins that does not have a suboptimal initiation of translation that affects partial exon skipping (see below) such that also the Rep 52 and/or 40 proteins are translated from the mRNA. Suitable nucleotide sequences encoding an mRNA, translation of which in the cell produces at least one of parvoviral Rep 78 and 68 proteins for use in the instant invention can be defined as a nucleotide sequence: a) that encodes a polypeptide comprising an amino acid sequence that has at least 50, 60, 70, 80, 88, 89, 90, 95, 97, 98, or 99% sequence identity with the amino acid sequence of SEQ ID NO. 18; b) that has at least 50, 60, 70, 80, 81, 82, 85, 90, 95, 97, 98, or 99% sequence identity with the nucleotide sequence of positions 11 - 1876 of SEQ ID NO. 19; c) the complementary strand of which hybridises to a nucleic acid molecule sequence of (a) or (b); and d) nucleotide sequences the sequence of which differs from the sequence of a nucleic acid molecule of (c) due to the degeneracy of the genetic code. It is understood that these Rep 78/60 coding sequence may or may not encode a suboptimal initiation of translation.

The first nucleic acid construct thus further comprises a second expression cassette for expression of the parvoviral Rep 52 and/or 40 proteins. The second expression cassette comprises a second promoter operably linked to a nucleotide sequence encoding an mRNA, translation of which in the cell produces at least one of parvoviral Rep 52 and 40 proteins.

In a preferred embodiment, the second expression cassette comprises a second promoter operably linked to a nucleotide sequence encoding an mRNA, translation of which in the cell produces only at least one of parvoviral Rep 52 and 40 proteins. Thereby it is understood that the nucleotide sequence encoding the parvoviral Rep 52 and/or 40 proteins is not part of a larger coding sequence that also encodes the parvoviral Rep 78 and/or 68 proteins. Preferably the nucleotide sequence encoding an mRNA, translation of which in the cell produces only at least one of parvoviral Rep 52 and 40 proteins comprises an open reading frame that consists of the amino acid sequence from the translation initiation codon to the most C-terminal amino acid of the at least one of parvoviral Rep 52 and 40 proteins, more preferably, the open reading frame is the only open reading frame comprised in the nucleotide sequence encoding an mRNA. Suitable nucleotide sequences encoding an mRNA, translation of which in the cell produces only at least one of parvoviral Rep 52 and 40 proteins for use in the instant invention can be defined as a nucleotide sequence: a) that encodes a polypeptide comprising an amino acid sequence that has at least 50, 60, 70, 80, 88, 89, 90, 95, 97, 98, or 99% sequence identity with the amino acid sequence of SEQ ID NO. 20; b) that has at least 50, 60, 70, 80, 81, 82, 85, 90, 95, 97, 98, or 99% sequence identity with the nucleotide sequence of any one of SEQ ID NO’s 21 - 25; c) the complementary strand of which hybridises to a nucleic acid molecule sequence of (a) or (b); and, d) nucleotide sequences the sequence of which differs from the sequence of a nucleic acid molecule of (c) due to the degeneracy of the genetic code.

Preferably, the nucleotide sequence encodes parvovirus Rep proteins that are required and sufficient for parvoviral vector production in insect cells.

In one embodiment, possible false translation initiation sites in the Rep protein coding sequences, other than the Rep78 and Rep52 translation initiation sites are eliminated. In one embodiment, putative splice sites that may be recognised in insect cells are eliminated from the Rep protein coding sequences. Elimination of these sites will be well understood by an artisan of skill in the art.

In a further embodiment, the at least one of parvoviral Rep 78 and 68 proteins and the at least one of parvoviral Rep 52 and 40 proteins comprise a common amino acid sequence comprising the amino acid sequence from the second amino acid to the most C-terminal amino acid of the at least one of parvoviral Rep 52 and 40 proteins, wherein the common amino acid sequences of the at least one of parvoviral Rep 78 and 68 proteins and the at least one of parvoviral Rep 52 and 40 proteins are at least 90, 91, 92, 93, 94, 95, 96, 97, 98, 99 or 100% identical, and wherein the nucleotide sequence encoding the common amino acid sequence of the at least one of parvoviral Rep 78 and 68 proteins and the nucleotide sequence encoding the common amino acid sequences of the at least one of parvoviral Rep 52 and 40 proteins are less than 90, 89, 88, 87, 86, 85, 84, 83, 82, 81, 80, 79, 78, 77, 76, 75, 74, 73, 72, 71, 70, 69, 68, 67, 66, 60% identical.

In one embodiment, the nucleotide sequence encoding the common amino acid sequence of the at least one of parvoviral Rep 78 and 68 proteins has an improved codon usage bias for the cell as compared to the nucleotide sequence encoding the common amino acid sequences of the at least one of parvoviral Rep 52 and 40, or wherein the nucleotide sequence encoding the common amino acid sequence of the at least one of parvoviral Rep 52 and 40 proteins has an improved codon usage bias for the cell as compared to the nucleotide sequence encoding the common amino acid sequences of the at least one of parvoviral Rep 78 and 68 proteins. In a further embodiment, the difference in codon adaptation index between the nucleotide sequences coding for the common amino acid sequences in the at least one of parvoviral Rep 78 and 68 proteins and the at least one of parvoviral Rep 52 and 40 proteins is at least 0.2.

The adaptiveness of a nucleotide sequence encoding the common amino acid sequence to the codon usage of the host cell can be expressed as codon adaptation index (CAI). Preferably the codon usage is adapted to the insect cell wherein Rep proteins with the common amino acid sequence are expressed. Usually this will be a cell of the genus Spodoptera, more preferably a Spodoptera frugiperda cell. The codon usage is thus preferably adapted to Spodoptera frugiperda or to an Autographa californica nucleopolyhedrovirus (AcMNPV) infected cell. A codon adaptation index is herein defined as a measurement of the relative adaptiveness of the codon usage of a gene towards the codon usage of highly expressed genes. The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (Sharp and Li , 1987, Nucleic Acids Research 15: 1281-1295; also see: Kim et al., Gene. 1997, 25 199:293-301; zur Megede et al., Journal of Virology, 2000, 74: 2628-2635).

Preferably, the difference in codon adaptation index between the nucleotide sequences coding for the common amino acid sequences in the at least one of parvoviral Rep 78 and 68 proteins and the at least one of parvoviral Rep 52 and 40 proteins is at least 0.1, 0.2, 0.3, 0.4, 0.5, 0.6, 0.7 or 0.8, whereby more preferably, the CAI of the nucleotide sequence coding forthe common amino acid sequence in the at least one of parvoviral Rep 52 and 40 proteins is at least 0.5, 0.6, 0.7, 0.8, 0.9 or 1.0.

Therefore, in an alternative embodiment, the at least one of parvoviral Rep 78 and 68 proteins and the at least one of parvoviral Rep 52 and 40 proteins comprise a common amino acid sequence comprising the amino acid sequence from the second amino acid to the most C-terminal amino acid of the at least one of parvoviral Rep 52 and 40 proteins, wherein the common amino acid sequences of the at least one of parvoviral Rep 78 and 68 proteins and the at least one of parvoviral Rep 52 and 40 proteins are at least 90% identical, and wherein the nucleotide sequence encoding the common amino acid sequence of the at least one of parvoviral Rep 78 and 68 proteins and the nucleotide sequence encoding the common amino acid sequences of the at least one of parvoviral Rep 52 and 40 proteins are less than 90% identical, and the nucleotide sequence encoding the common amino acid sequence of the at least one of parvoviral Rep 78 and 68 proteins has an improved codon usage bias for the cell as compared to the nucleotide sequence encoding the common amino acid sequences of the at least one of parvoviral Rep 52 and 40, or wherein the nucleotide sequence encoding the common amino acid sequence of the at least one of parvoviral Rep 52 and 40 proteins has an improved codon usage bias for the cell as compared to the nucleotide sequence encoding the common amino acid sequences of the at least one of parvoviral Rep 78 and 68 proteins, wherein preferably, the difference in codon adaptation index between the nucleotide sequences coding for the common amino acid sequences in the at least one of parvoviral Rep 78 and 68 proteins and the at least one of parvoviral Rep 52 and 40 proteins is at least 0.2. Codon optimization of the parvoviral Rep protein is discussed in more detail hereafter.

Temperature optimization of the parvoviral Rep protein refers to use the optimal condition with respect to both the temperature at which the insect cell will grow and Rep is functioning. A Rep protein may for example be optimally active at 37° C., whereas an insect cell may grow optimally at 28° C. A temperature at which both the Rep protein is active and the insect cell grows may be 30° C. In a preferred embodiment, the optimized temperature is more than 27, 28, 29, 30, 31, 32, 33, 34 or 35° C. and/or less than 37, 36, 35, 34, 33, 32, 31, 30 or 29° C.

As will be understood by the skilled person in the art, the full virion:empty virion ratio may also be improved by attenuated Cap expression, for example by means of a weaker promoter, as compared to moderate to high Rep expression.

In one embodiment, the nucleotide sequence encoding an mRNA, translation of which in the cell produces only at least one of parvoviral Rep 78 and 68 proteins, comprises an intact parvoviral p19 promoter, as is e.g. present in the native parvoviral nucleotide sequence encoding the parvoviral Rep 78 and 68 proteins.

In one embodiment, the first and second expression cassettes in the first nucleic acid construct are optimised to obtain a desired molar ratio of Rep78 to Rep52 in the (insect) cell. Preferably, the first nucleic acid construct produces a molar ratio of Rep78 to Rep52 in the range of 1:10 to 10:1, 1:5 to 5:1, or 1:3 to 3:1 in the (insect) cell. More preferably, the first nucleic acid construct produces a molar ratio of Rep78 to Rep52 that is at least 1:2, 1:3, 1:5 or 1:10. The molar ration of the Rep78 and Rep52 may be determined by means of Western blotting, preferably using a monoclonal antibody that recognizes a common epitope of both Rep78 and Rep52, or using e.g. a mouse anti-Rep antibody (303.9, Progen, Germany; dilution 1:50).

A desired molar ratio of Rep78 to Rep52 can be obtained by the choice of the promoters in respectively the first and second expression cassettes as herein further described below. Alternatively or in combination, the desired molar ratio of Rep78 to Rep52 can be obtained by using means to reduce the steady state level of the at least one of parvoviral Rep 78 and 68 proteins.

Thus, in one embodiment, the nucleotide sequence encoding the mRNA for the at least one of parvoviral Rep 78 and 68 proteins comprises a modification that affects a reduced steady state level of the at least one of parvoviral Rep 78 and 68 proteins. The reduced steady state condition can be achieved in example by truncating the regulation element or upstream promoter (Urabe et al., supra, Dong et al., supra), adding protein degradation signal peptide, such as the PEST or ubiquitination peptide sequence, substituting the start codon into a more suboptimal one, or by introduction of an artificial intron as described in WO 2008/024998.

In a preferred embodiment, the nucleotide sequence encoding at least one of parvoviral Rep78 and 68 proteins comprises an open reading frame that starts with a suboptimal translation initiation codon. The suboptimal initiation codon preferably is an initiation codon that affects partial exon skipping. Partial exon skipping is herein understood to mean that at least part of the ribosomes do not initiate translation at the suboptimal initiation codon of the Rep78 protein but may initiate at an initiation codon further downstream, whereby preferably the (first) initiation codon further downstream is the initiation codon of the Rep52 protein. Alternatively, the nucleotide sequence encoding at least one of parvoviral Rep78 and 68 proteins comprises an open reading frame that starts with a suboptimal translation initiation codon and has no initiation codons further downstream. The suboptimal initiation codon preferably affects partial exon skipping upon expression of the nucleotide sequence in an insect cell.

The term “suboptimal initiation codon” herein not only refers to the tri-nucleotide initiation codon itself but also to its context. Thus, a suboptimal initiation codon may consist of an “optimal” ATG codon in a suboptimal context, e.g. a non-Kozak context. However, more preferred are suboptimal initiation codons wherein the tri-nucleotide initiation codon itself is suboptimal, i.e. is not ATG. Suboptimal is herein understood to mean that the codon is less efficient in the initiation of translation in an otherwise identical context as compared to the normal ATG codon. Preferably, the efficiency of suboptimal codon is less than 90, 80, 60, 40 or 20% of the efficiency of the normal ATG codon in an otherwise identical context. Methods for comparing the relative efficiency of initiation of translation are known per se to the skilled person. Preferred suboptimal initiation codons may be selected from ACG, TTG, CTG, and GTG. More preferred is ACG. A nucleotide sequence encoding parvovirus Rep proteins, is herein understood as a nucleotide sequence encoding the non-structural Rep proteins that are required and sufficient for parvoviral vector production in insect cells such the Rep78 and Rep52 proteins.

### Capsid Proteins

A nucleotide sequence encoding a parvoviral capsid (Cap) protein is herein understood to comprise nucleotide sequences encoding one or more of the three parvoviral capsid proteins, VP1, VP2 and VP3. The parvovirus nucleotide sequence preferably is from a dependovirus, more preferably from a human or simian adeno-associated virus (AAV) and most preferably from an AAV which normally infects humans (e.g., serotypes 1, 2, 3A, 3B, 4, 5, 6, 7, 8, 9, 10, 11, 12 or 13) or primates (e.g., serotypes 1 and 4), of which the nucleotide and amino acid sequences are listed in Lubelski et al., US2017356008, which is incorporated herein in its entirety by reference. Hence, the nucleic acid construct according to the present invention can comprise an entire open reading frame for AAV capsid proteins as disclosed by Lubelski et al., US2017356008. Alternatively, the sequence can be man-made, for example, the sequence may be a hybrid form or may be codon optimized, such as for example by codon usage of AcmNPv or Spodoptera frugiperda. For example, the capsid sequence may be composed of the VP2 and VP3 sequences of AAV1 whereas the remainder of the VP1 sequence is of AAV5. A preferred capsid protein is AAV5 or AAV8, as provided in SEQ ID NO. 26, as listed in Lubelski et al. US2017356008. Thus, in a preferred embodiment, the AAV capsid proteins are AAV serotype 5 or AAV serotype 8 capsid proteins that have been modified according to the invention. More preferably, the AAV capsid proteins are AAV serotype 5 capsid proteins that have been modified according to the invention. It is understood that the exact molecular weights of the capsid proteins, as well as the exact positions of the translation initiation codons may differ between different parvoviruses. However, the skilled person will know how to identify the corresponding position in nucleotide sequence from other parvoviruses than AAV5. Alternatively, the sequence encoding AAV capsid proteins is a man-made sequence, for example as a result of directed evolution experiments. This can include generation of capsid libraries via DNA shuffling, error prone PCR, bioinformatic rational design, site saturated mutagenesis. Resulting capsids are based on the existing serotypes but contain various amino acid or nucleotide changes that improve the features of such capsids. The resulting capsids can be a combination of various parts of existing serotypes, “shuffled capsids” or contain completely novel changes, i.e. additions, deletions or substitutions of one or more amino acids or nucleotides, organized in groups or spread over the whole length of gene or protein. See for example Schaffer and Maheshri; Proceedings of the 26^{th} Annual International Conference of the IEEE EMBS San Francisco, CA, USA; September 1-5, 2004, pages 3520-3523; Asuri et al., 2012, Molecular Therapy 20(2):329-3389; Lisowski et al., 2014, Nature 506(7488):382-386, herein incorporated by reference.

In a preferred embodiment of the invention, the open reading frame encoding a VP1 capsid protein starts with non-canonical translation initiation codon selected from the group consisting of: ACG, ATT, ATA, AGA, AGG, AAA, CTG, CTT, CTC, CTA, CGA, CGC, TTG, TAG and GTG. Preferably, the non-canonical translation initiation codon is selected from the group consisting of GTG, CTG, ACG, and TTG, more preferably the non-canonical translation initiation codon is CTG.

The nucleotide sequence of the invention for expression of the AAV capsid proteins further preferably comprises at least one modification of the nucleotide sequence encoding AAV VP1 capsid protein selected from among a G at nucleotide position 12, an A at nucleotide position 21, and a C at nucleotide position 24 of the VP1 open reading frame, wherein the nucleotide positions correspond to the nucleotide positions of the wild-type nucleotide sequences. A “potential/possible false start site” or “potential/possible false translation initiation codon” is herein understood to mean an in-frame ATG codon located in the coding sequence of the capsid protein(s). Elimination of possible false start sites for translation within the VP1 coding sequences of other serotypes will be well understood by an artisan of skill in the art, as will be the elimination of putative splice sites that may be recognized in insect cells. For example, the modification of the nucleotide at position 12 is not required for recombinant AAV5, since the nucleotide T is not giving rise to a false ATG codon. Specific examples of a nucleotide sequence encoding parvovirus capsid proteins are given in SEQ ID NOs. 27 to 29. Nucleotide sequences encoding parvoviral Cap and/or Rep proteins of the invention may also be defined by their capability to hybridise with the nucleotide sequences of SEQ ID NOs. SEQ ID NOs. 27 to 29 and 21 to 25, respectively, under moderate, or preferably under stringent hybridisation conditions.

The capsid protein coding sequences may be present in various forms. E.g. separate coding sequences for each of the capsid proteins VP1, -2 and -3 may be used, whereby each coding sequence is operably linked to expression control sequences for expression in an insect cell. More preferably, however, the second expression cassette comprises a nucleotide sequence comprising a single open reading frame encoding all three of the parvoviral (AAV) VP1, VP2, and VP3 capsid proteins, wherein the initiation codon for translation of the VP1 capsid protein is a suboptimal initiation codon that is not ATG as e.g. described by Urabe et al., (2002, supra) and in WO2007/046703. A suboptimal initiation codon for the VP1 capsid protein may be as defined above for the Rep78 protein. More preferred suboptimal initiation codons for the VP1 capsid protein may be selected from ACG, TTG, CTG and GTG, of which CTG and ACG are most preferred.

In an alternative embodiment, the second expression cassette comprises a nucleotide sequence comprising a single open reading frame encoding all three of the parvoviral (AAV) VP1, VP2, and VP3 capsid proteins, wherein the initiation codon for translation of the VP1 capsid protein is ATG and wherein the mRNA coding for the VP1 capsid protein as encoded in the nucleotide sequence comprises an alternative start codon which is out of frame with the open reading frame the VP1 capsid protein (as described in WO2019/016349). Preferably, the alternative start codon is selected from the group consisting of CTG, ATG, ACG, TTG, GTG, CTC and CTT, of which ATG is preferred. Preferably, the AAV capsid proteins are AAV5 serotype capsid proteins. Preferably in this embodiment, the nucleotide sequence comprises an alternative open reading frame starting with the alternative start codon that encompasses said ATG translation initiation codon for VP1, whereby preferably, the alternative open reading frame following the alternative start codon encodes a peptide of up to 20 amino acids.

The nucleotide sequence comprised in the second expression cassette for expression of the capsid proteins may further comprise one or more modifications as described in WO2007/046703. Various further modifications of VP coding regions are known to the skilled artisan which could either increase yield of VP and virion or have other desired effects, such as altered tropism or reduce antigenicity of the virion. These modifications are within the scope of the present invention.

In one embodiment, the expression of VP1 is increased as compared to the expression of VP2 and VP3. VP1 expression may be increased by supplementation of VP1, by introduction into the insect cell of a single vector comprising nucleotide sequences for the VP1 as has been described in WO 2007/084773.

Typically, in a method of the invention, at least one open reading frame comprising nucleotide sequences encoding the VP1, VP2 and VP3 capsid proteins or at least one open reading frame, comprising an open reading frame comprising nucleotide sequences encoding at least one of the Rep78 and Rep68 proteins. In one embodiment, the VP1, VP2 and VP3 capsid proteins or at least one open reading frame comprising an open reading frame comprising nucleotide sequences encoding at least one of the Rep78 and Rep68 proteins does not comprise an artificial intron (or a sequence derived from an artificial intron). That is to say, at least open reading frames used to encode Rep or VP proteins will not comprise an artificial intron. By artificial intron is meant to be an intron which would not naturally occur in an adeno-associated virus Rep or Cap sequence, for example an intron which has been engineered so as to permit functional splicing within an insect cell. An artificial intron in this context therefore encompass wild-type insect cell introns. An expression cassette of the invention may comprise native truncated intron sequence (by native is meant sequence naturally occurring in an adeno-associated virus) - such sequences are not intended to fall within the meaning of artificial intron as defined herein.

In the invention, one possibility is that no open reading frame comprising nucleotide sequences encoding the VP1, VP2 and VP3 capsid proteins and/or no open reading frame comprising nucleotide sequences encoding at least one of the Rep78 and Rep68 proteins comprises an artificial intron.

### Promoters

Preferably, a nucleotide sequence of the invention encoding the AAV proteins is operably linked to expression control sequences for expression in an insect cell. These expression control sequences will at least include a promoter that is active in insect cells.

A suitable promote to be used as third and/or fourth promoter, for controlling transcription of the nucleotide sequence of the invention encoding of the parvoviral capsid proteins, is e.g. the polyhedron promoter (poIH), such a poIH promoter provided as SEQ ID NO. 30, and shortened version thereof SEQ ID NO. 31, as disclosed in Lubelski et al. US2017356008. However, other promoters that are active in insect cells and that may be selected according to the invention are known in the art, e.g. an polyhedrin (poIH) promoter, p10 promoter, p35 promoter, 4xHsp27 EcRE+minimal Hsp70 promoter, deltaE1 promoter, E1 promoter or IE-1 promoter and further promoters described in the above references. In one embodiment, the promoter for transcription of the nucleotide sequence of the invention encoding of the AAV capsid proteins is p10 or poIH. In a further embodiment, the promoter for transcription of the nucleotide sequence of the invention encoding of the AAV capsid proteins is p10. In an alternative embodiment, the promoter for transcription of the nucleotide sequence of the invention encoding of the AAV capsid proteins is poIH.

These above promoters can also be used as first and second promoter for controlling transcription of the nucleotide sequence of the invention encoding of the parvoviral Rep proteins. In one embodiment, the first promoter is a constitutive promoter. As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer. A “tissue specific” promoter is only active in specific types of tissues or cells. A “cryptic promoter” is an epigenetically silenced promoter which may be activated.

In a preferred embodiment, the ratio of expression of the Rep78 versus the Rep52 protein is regulated by one or more of the following: (a) the second promoter is stronger than the first promoter, as e.g. determined by reporter gene expression (e.g. luciferase or SEAP), or northern blot; (b) the presence of nucleotide spacer or more and/or stronger enhancer elements at upstream of the second expression cassette as compared to the first expression cassette; (c) the nucleotide sequence coding for the parvoviral Rep52 protein has a higher codon adaptation index as compared to the nucleotide sequence coding for the Rep78 protein; (d) temperature optimization of the parvoviral Rep protein; and variant Rep proteins with one or more alterations in the amino acid sequence as compared to a corresponding wild-type Rep protein and wherein the one or more amino acid alteration result in increases in the activity of the Rep function as assessed by detecting increased AAV production in insect cells. Methods for generation, selection and/or screening of variant Rep proteins with increased activity of Rep function as assessed by detecting increased AAV production in insect cells may be obtained by adaptation to insect cells of the methods described in US20030134351 for obtaining variant Rep proteins with increased function with respect to AAV production in mammalian cells. Variant Rep proteins with one or more alterations in the amino acid sequence as compared to a corresponding wild-type Rep protein are herein understood to include Rep proteins with one or more amino acid substitutions, insertions and/or deletions in the variant amino acid sequence as compared to the amino acid sequence of a corresponding wild type Rep protein.

The second promoter being stronger than the first promoter means that more nucleotide sequences encoding for a Rep52 protein are expressed than nucleotide sequences encoding for a Rep78 protein. An equally strong promoter may be used, since expression of Rep52 protein will then be increased as compared to expression of Rep78 protein. The strength of the promoter may be determined by the expression that is obtained under conditions that are used in the method of the invention. In one embodiment, at least one of the second, third and fourth promoters is an inducible promoter, preferably selected from poIH and p10. In a further embodiment, the inducible promoter is a viral promoter that is induced at a later stage in the virus’ infection cycle, preferably the viral promoter that is induced at least 24 hours after transfection or infection of the cell with the virus.

In one embodiment, the first promoter is selected from a deltaE1 promoter or an E1 promoter; and, the second, third and fourth promoters are selected from a poIH promoter or a p10 promoter. In a further embodiment, the first promoter is deltaE1 and the second promoter is poIH.

Using the same baculovirus promoter twice on the same baculovirus construct to drive separate AAV genes can result in competition between the promoters. This competition will result in decreased expression of the Cap and Rep genes and thereby reduce AAV yields. Close proximity of similar elements within an expression cassette can potentially enhance this effect. Expression of attenuated genes can be improved by using a stronger start codon or exchanging the promoter driving the Capsid protein (e.g. poIH to P10). Therefore, in a preferred embodiment the first, second and third promoters are different promoters, more preferably, the first, second, third and fourth promoters are different promoters.

### Enhancer

An “enhancer element” or “enhancer” is meant to define a sequence which enhances the activity of a promoter (i.e. increases the rate of transcription of a sequence downstream of the promoter) which, as opposed to a promoter, does not possess promoter activity, and which can usually function irrespective of its location with respect to the promoter (i.e. upstream, or downstream of the promoter). Enhancer elements are well-known in the art. Non-limiting examples of enhancer elements (or parts thereof) which could be used in the present invention include baculovirus enhancers and enhancer elements found in insect cells. It is preferred that the enhancer element increases in a cell the mRNA expression of a gene, to which the promoter it is operably linked, by at least 25%, more preferably at least 50%, even more preferably at least 100%, and most preferably at least 200% as compared to the mRNA expression of the gene in the absence of the enhancer element. mRNA expression may be determined for example by quantitative RT-PCR.

Herein it is preferred to use an enhancer element to enhance the expression of parvoviral Rep protein. Thus, in one embodiment, at least one expression cassette comprises at least one baculovirus enhancer element and/or at least one ecdysone responsive element, wherein preferable the enhancer element is selected from the group consisting of hr1, hr2, hr3, hr4 and hr5. Preferably the enhancer element is responsive to a baculoviral immediate-early protein (IE1) or its splice variant (IE0), such as a baculoviral homologous region (hr) enhancer element, wherein preferably the baculovirus is Autographa californica multicapsid nucleopolyhedrovirus. IE1 is a highly conserved, 67-kDa DNA binding protein that transactivates baculovirus early gene promoters and supports late gene expression in plasmid transfection assays (see e.g. Olson et al., 2002, J Virol., 76:9505-9515). AcMNPV IE1 possesses separable domains that contribute to promoter transactivation and DNA binding. The N-terminal half of this 582-residue phosphoprotein contains transcriptional stimulatory domains from residue 8 to 118 and 168 to 222. IE1 binds to the ~28-bp imperfect palindrome (28-mer) that constitutes repetitive sequences within multiple homologous regions (hrs) found dispersed throughout the AcMNPV genome. The hr 28-mer is the minimal sequence motif required for IE1-mediated enhancer and origin-specific replication functions.

In one embodiment, the hr enhancer element is an hr enhancer element other than hr2-0.9 US 2012/100606 A1). In a further embodiment, the hr enhancer element is selected from the group consisting of hr1, hr3, hr4b and hr5, of which hr4b and hr5 are preferred, of which hr4b is most preferred. In an alternative embodiment, the hr enhancer element is a variant hr enhancer element, such as e.g. a non-naturally occurring designed element. The variant hr enhancer element preferably comprises at least one copy of the hr 28-mer sequence CTTTACGAGTAGAATTCTACGCGTAAAA (SEQ ID NO. 32) and/or at least one copy of a of a sequence of which at least 18, 20, 21, 22, 23, 24, 25, 26, or 27 nucleotides are identical to sequence CTTTACGAGTAGAATTCTACGCGTAAAA (SEQ ID NO. 32) and which preferably binds to the baculoviral IE1 protein, more preferably to the AcMNPV IE1 protein. The variant hr enhancer element is further preferably functionally defined in that when the variant element is operably linked to an expression cassette comprising a reporter gene operably linked to the poIH promoter, a) under non-inducing conditions, the cassette with the variant element produces less reporter transcript than an otherwise identical expression cassette which comprises the hr2-0.9 element instead of the variant element, or the cassette with the variant element produces less than a factor 1.1, 1.2, 1.5, 2, 5 or 10 of the amount reporter transcript produced by an otherwise identical expression cassette which comprises the hr4b element instead of the variant element; and b) under inducing conditions, the cassette with the variant element produces at least 50, 60, 70, 80, 90 or 100% of the amount of reporter transcript produced by an otherwise identical expression cassette which comprises the hr4b or the hr2-0.9 element instead of the variant element. Non-inducing conditions are understood as condition in which there is no IE1 protein present in the cell wherein the cassettes are tested and inducing conditions are understood to be conditions wherein sufficient IE1 protein is present to obtain maximal reporter expression with the reference cassettes comprising the hr4b or the hr2-0.9 element. Binding of the variant hr enhancer element to the baculoviral IE1 protein can be assayed by using a mobility shift assay as e.g. described by Rodems and Friesen (J Virol. 1995; 69(9):5368-75).

### Viral Vectors

The present invention relates to the use of parvoviruses, in particular dependoviruses such as infectious human or simian AAV, and the components thereof (e.g., a parvovirus genome) for use as vectors for introduction and/or expression of nucleic acids in mammalian cells, preferably human cells. In particular, the invention relates to improvements in productivity of such parvoviral vectors when produced in insect cells.

Productivity in this context encompasses improvements in production titres and improvements in the quality of the resulting product, for example a product which has improved a total:full ratio (a measure of the number of particles which comprise nucleic acid). That is to say, the final product may have an increased proportion of filled particles, where filled implies that the particle comprises nucleic acid.

A “parvoviral vector” is defined as a recombinantly produced parvovirus or parvoviral particle that comprises a polynucleotide to be delivered into a host cell, either in vivo, ex vivo or in vitro. Examples of parvoviral vectors include e.g., adeno-associated virus vectors. Herein, a parvoviral vector construct refers to the polynucleotide comprising the viral genome or part thereof, and a transgene. Viruses of the Parvoviridae family are small DNA viruses. The family Parvoviridae may be divided between two subfamilies: the Parvovirinae, which infect vertebrates, and the Densovirinae, which infect invertebrates, including insects. Members of the subfamily Parvovirinae are herein referred to as the parvoviruses and include the genus Dependovirus. As may be deduced from the name of their genus, members of the Dependovirus are unique in that they usually require coinfection with a helper virus such as adenovirus or herpes virus for productive infection in cell culture. The genus Dependovirus includes AAV, which normally infects humans (e.g., serotypes 1, 2, 3A, 3B, 4, 5, 6, 7, 8, 9, 10, 11, 12 and 13) or primates (e.g., serotypes 1 and 4), and related viruses that infect other warm-blooded animals (e.g., bovine, canine, equine, and ovine adeno-associated viruses). Further information on parvoviruses and other members of the Parvoviridae is described in Kenneth I. Berns, “Parvoviridae: The Viruses and Their Replication,” Chapter 69 in Fields Virology (3d Ed. 1996). While it is understood that the invention is not limited to AAV but may equally be applied to other parvoviruses, for convenience, the present invention is further exemplified and described herein by reference to AAV. Therefore, in one embodiment, the at least one of parvoviral Rep 78 and 68 proteins, the at least one of parvoviral Rep 52 and 40 proteins, the parvoviral VP1, VP2, and VP3 capsid proteins and the at least one parvoviral inverted terminal repeat sequence are from an AAV, preferably of a serotype that infect humans.

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences forthe non-structural replication (Rep) proteins and the structural viral particle (VP) proteins. The VP proteins (VP1, -2 and -3) form the capsid. The terminal 145 nt ITRs are self-complementary and are organized so that an energetically stable intramolecular duplex forming a T-shaped hairpin may be formed. These hairpin structures function as an origin for viral DNA replication, serving as primers for the cellular DNA polymerase complex. Following wildtype (wt) AAV infection in mammalian cells the Rep genes (i.e. Rep78 and Rep52) are expressed from the P5 promoter and the P19 promoter, respectively, and both Rep proteins have a function in the replication and packaging of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production. The three capsid proteins, VP1, VP2 and VP3 are expressed from a single VP reading frame from the p40 promoter. wtAAV infection in mammalian cells relies for the capsid proteins production on a combination of alternate usage of two splice acceptor sites and the suboptimal utilization of an ACG initiation codon for VP2.

A “recombinant parvoviral or AAV vector” (or “rAAV vector”) herein refers to a vector comprising one or more polynucleotide sequences of interest, genes of interest or “transgenes” that is/are flanked by at least one parvoviral or AAV inverted terminal repeat sequence (lTR). Preferably, the transgene(s) is/are flanked by ITRs, one on each side of the transgene(s). Such rAAV vectors can be replicated and packaged into infectious viral particles when present in an insect host cell that is expressing AAV rep and cap gene products (i.e. AAV Rep and Cap proteins). When an rAAV vector is incorporated into a larger nucleic acid construct (e.g. in a chromosome or in another vector such as a plasmid or baculovirus used for cloning or transfection), then the rAAV vector is typically referred to as a “pro-vector” which can be “rescued” by replication and encapsidation in the presence of AAV packaging functions and necessary helper functions.

It is preferred that the nucleotide sequence of (ii) comprises an open reading frame comprising nucleotide sequences encoding at least one of the Rep78 and Rep68 proteins. Preferably, the nucleotide sequences are of the same serotype. More preferably, the nucleotide sequences differ from each other in that they may be either codon optimized, AT-optimized or GC-optimized, to minimize or prevent recombination. Preferably, the first expression cassette comprises two nucleotide sequences encoding a parvoviral Rep protein, i.e., a first nucleotide sequence and a second nucleotide sequence. Preferably, the difference in the first and the second nucleotide sequence coding for the common amino acid sequences of a parvoviral Rep protein is maximised (i.e. the nucleotide identity is minimised) by one or more of: a) changing the codon bias of the first nucleotide sequence coding for the parvoviral Rep common amino acid sequence; b) changing the codon bias of the second nucleotide sequence coding for the parvoviral Rep common amino acid sequence; c) changing the GC-content of the first nucleotide sequence coding for the common amino acid sequence; and d) changing the GC-content of the second nucleotide sequence coding for the common amino acid sequence. Codon optimisation may be performed on the basis of the codon usage of the insect cell used in the method of the invention, preferably Spodoptera frugiperda, as may be found in a codon usage database (see e.g. http://www.kazusa.or.jp/codon/). Suitable computer programs for codon optimisation are available to the skilled person (see e.g. Jayaraj et al., 2005, Nucl. Acids Res. 33(9):3011-3016; and on the internet). Alternatively the optimisations can be done by hand, using the same codon usage database.

### Transgene

In one embodiment the invention relates to a cell, wherein the nucleotide sequence comprising the transgene flanked by the parvoviral inverted terminal repeat sequence is present on a second nucleic acid construct (that is different from the first nucleic acid construct). In a preferred embodiment, the nucleotide sequence comprising the transgene flanked by the parvoviral inverted terminal repeat sequence is present on a second nucleic acid construct (that is different from the first nucleic acid construct).

In the context of the invention “at least one parvoviral inverted terminal repeat nucleotide sequence” is understood to mean a palindromic sequence, comprising mostly complementary, symmetrically arranged sequences also referred to as “A,” “B,” and “C” regions. The ITR functions as an origin of replication, a site having a “cis” role in replication, i.e. being a recognition site for trans acting replication proteins, such as e.g. Rep 78 (or Rep68), which recognize the palindrome and specific sequences internal to the palindrome. One exception to the symmetry of the ITR sequence is the “D” region of the ITR. It is unique (not having a complement within one ITR). Nicking of single-stranded DNA occurs at the junction between the A and D regions. It is the region where new DNA synthesis initiates. The D region normally sits to one side of the palindrome and provides directionality to the nucleic acid replication step. A parvovirus replicating in a mammalian cell typically has two ITR sequences. It is, however, possible to engineer an ITR so that binding sites on both strands of the A regions and D regions are located symmetrically, one on each side of the palindrome. On a double-stranded circular DNA template (e.g., a plasmid), the Rep78- or Rep68-assisted nucleic acid replication then proceeds in both directions and a single ITR suffices for parvoviral replication of a circular vector. Thus, one ITR nucleotide sequence can be used in the context of the present invention. Preferably, however, two or another even number of regular ITRs are used. Most preferably, two ITR sequences are used. A preferred parvoviral ITR is an AAV ITR. More preferably AAV2 ITRs are used. For safety reasons it may be desirable to construct a recombinant parvoviral (rAAV) vector that is unable to further propagate after initial introduction into a cell in the presence of a second AAV. Such a safety mechanism for limiting undesirable vector propagation in a recipient may be provided by using rAAV with a chimeric ITR as described in US2003148506.

The term “flanked” with respect to a sequence that is flanked by another element(s) herein indicates the presence of one or more of the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence that is “flanked” by two other elements (e.g. ITRs), indicates that one element is located 5′ to the sequence and the other is located 3′ to the sequence; however, there may be intervening sequences there between. In a preferred embodiment the nucleotide sequence of (iv) is flanked on either side by parvoviral inverted terminal repeat nucleotide sequences.

In the embodiments of the invention, the nucleotide sequence comprising the transgene (encoding a gene product of interest) that is flanked by at least one parvoviral ITR sequence preferably becomes incorporated into the genome of a recombinant parvoviral (rAAV) vector produced in the insect cell. Preferably, the transgene encodes a gene product of interest for expression in a mammalian cell. Preferably, the nucleotide sequence comprising the transgene is flanked by two parvoviral (AAV) ITR nucleotide sequences and wherein the transgene is located in between the two parvoviral (AAV) ITR nucleotide sequences. Preferably, the nucleotide sequence encoding a gene product of interest (for expression in the mammalian cell) will be incorporated into the recombinant parvoviral (rAAV) vector produced in the insect cell if it is located between two regular ITRs, or is located on either side of an ITR engineered with two D regions.

AAV sequences that may be used in the present invention forthe production of a recombinant AAV virion in insect cells can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession numberAF043303; GenBank Accession numberAF085716; Chlorini et al. (1997, J. Vir. 71 : 6823-33) ; Srivastava et al. (1983, J. Vir. 45 :555-64) ; Chlorini et al. (1999, J. Vir. 73:1309-1319); Rutledge et al. (1998, J. Vir. 72:309-319); and Wu et al. (2000, J. Vir. 74: 8635-47). AAV serotypes 1, 2, 3, 4 and 5 are preferred source of AAV nucleotide sequences for use in the context of the present invention. Preferably the AAV ITR sequences for use in the context of the present invention are derived from AAV1, AAV2, AAV4 and/or AAV7. Likewise, the Rep (Rep78/68 and Rep52/40) coding sequences are preferably derived from AAV1, AAV2, AAV4 and/or AAV7. The sequences coding for the VP1, VP2, and VP3 capsid proteins for use in the context of the present invention may however be taken from any of the known 42 serotypes, more preferably from AAV1, AAV2, AAV3, AAV4, AAV5, AAV6, AAV7, AAV8. AAV9, AAV10, AAV11, AAV12 or AAV13 or newly developed AAV-like particles obtained by e.g. capsid shuffling techniques and AAV capsid libraries, or from newly and synthetically designed, developed or evolved capsid, such as the Anc-80 capsid.

AAV Rep and ITR sequences are particularly conserved among most serotypes. The Rep78 proteins of various AAV serotypes are e.g. more than 89% identical and the total nucleotide sequence identity at the genome level between AAV2, AAV3A, AAV3B, and AAV6 is around 82% (Bantel-Schaal et al., 1999, J. Virol., 73(2):939-947). Moreover, the Rep sequences and ITRs of many AAV serotypes are known to efficiently cross-complement (i.e., functionally substitute) corresponding sequences from other serotypes in production of AAV particles in mammalian cells. US2003148506 reports that AAV Rep and ITR sequences also efficiently cross-complement other AAV Rep and ITR sequences in insect cells.

The AAV capsid proteins, also known as VP proteins, are known to determine the cellular tropism of the AAV virion. The VP protein-encoding sequences are significantly less conserved than Rep proteins and genes among different AAV serotypes. The ability of Rep and ITR sequences to cross-complement corresponding sequences of other serotypes allows for the production of pseudotyped rAAV particles comprising the capsid proteins of a serotype (e.g., AAV3) and the Rep and/or ITR sequences of another AAV serotype (e.g., AAV2). Such pseudotyped rAAV particles are a part of the present invention.

Modified “AAV” sequences also can be used in the context of the present invention, e.g. for the production of rAAV vectors in insect cells. Such modified sequences e.g. include sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAV5, AAV6, AAV7, AAV8, AAV9, AAV10, AAV11, AAV12 or AAV13 ITR, Rep, or VP can be used in place of wild-type AAV ITR, Rep, or VP sequences.

Although similar to other AAV serotypes in many respects, AAV5 differs from other human and simian AAV serotypes more than other known human and simian serotypes. In view thereof, the production of rAAV5 can differ from production of other serotypes in insect cells. Where methods of the invention are employed to produce rAAV5, it is preferred that one or more constructs comprising, collectively in the case of more than one construct, a nucleotide sequence comprising an AAV5 ITR, a nucleotide sequence comprises an AAV5 Rep coding sequence (i.e. a nucleotide sequence comprises an AAV5 Rep78). Such ITR and Rep sequences can be modified as desired to obtain efficient production of rAAV5 or pseudotyped rAAV5 vectors in insect cells. E.g., the start codon of the Rep sequences can be modified, VP splice sites can be modified or eliminated, and/or the VP1 start codon and nearby nucleotides can be modified to improve the production of rAAV5 vectors in the insect cell.

Typically, the gene product of interest, including ITRs, is 5,000 nucleotides (nt) or less in length. In another embodiment, an oversized DNA molecule, i.e. more than 5,000 nt in length, can be expressed in vitro or in vivo by using the AAV vector described by the present invention. An oversized DNA is here understood as a DNA exceeding the maximum AAV packaging limit of 5.5 kbp. Therefore, the generation of AAV vectors able to produce recombinant proteins that are usually encoded by larger genomes than 5.0 kb is also feasible.

The nucleotide sequence comprising the transgene as defined herein above may thus comprise a nucleotide sequence encoding a gene product of interest (for expression in the mammalian cell) or the nucleotide sequence targeting a gene of interest (for silencing said gene of interest in a mammalian cell), and may be located such that it will be incorporated into an recombinant parvoviral (rAAV) vector replicated in the insect cell. In the context of the invention it is understood that a particularly preferred mammalian cell in which the “gene product of interest” is to be expressed or silenced, is a human cell. Any nucleotide sequence can be incorporated for later expression in a mammalian cell transfected with the recombinant parvoviral (rAAV) vector produced in accordance with the present invention. The nucleotide sequence may e.g. encode a protein or it may express an RNAi agent, i.e. an RNA molecule that is capable of RNA interference such as, e.g. a shRNA (short hairpin RNA) or a siRNA (short interfering RNA). “siRNA” means a small interfering RNA that is a short-length double-stranded RNA that are not toxic in mammalian cells (Elbashir et al., 2001, Nature 411: 494-98; Caplen et al., 2001, Proc. Natl. Acad. Sci. USA 98: 9742-47). In a preferred embodiment, the nucleotide sequence comprising the transgene may comprise two coding nucleotide sequences, each encoding one gene product of interest for expression in a mammalian cell. Each of the two nucleotide sequences encoding a product of interest is located such that it will be incorporated into a recombinant parvoviral (rAAV) vector replicated in the insect cell.

The product of interest for expression in a mammalian cell may be a therapeutic gene product. A therapeutic gene product can be a polypeptide, or an RNA molecule (si/sh/miRNA), or other gene product that, when expressed in a target cell, provides a desired therapeutic effect. A desired therapeutic effect can for example be the ablation of an undesired activity (e.g. VEGF), the complementation of a genetic defect, the silencing of genes that cause disease, the restoration of a deficiency in an enzymatic activity or any other disease-modifying effect. Examples of therapeutic polypeptide gene products include, but are not limited to growth factors, factors that form part of the coagulation cascade, enzymes, lipoproteins, cytokines, neurotrophic factors, hormones and therapeutic immunoglobulins and variants thereof. Examples of therapeutic RNA molecule products include miRNAs effective in silencing diseases, including but not limited to polyglutamine diseases, dyslipidaemia or amyotrophic lateral sclerosis (ALS).

The diseases that can be treated using a recombinant parvoviral (rAAV) vector produced in accordance with the present invention are not particularly limited, other than generally having a genetic cause or basis. For example, the disease that may be treated with the disclosed vectors may include, but are not limited to, acute intermittent porphyria (AIP), age-related macular degeneration, Alzheimer’s disease, arthritis, Batten disease, Canavan disease, Citrullinemia type 1, Crigler Najjar, congestive heart failure, cystic fibrosis, Duchene muscular dystrophy, dyslipidemia, glycogen storage disease type I (GSD-I), hemophilia A, hemophilia B, hereditary emphysema, homozygous familial hypercholesterolemia (HoFH), Huntington’s disease (HD), Leber’s congenital amaurosis, methylmalonic academia, ornithine transcarbamylase deficiency (OTC), Parkinson’s disease, phenylketonuria (PKU), spinal muscular atrophy, paralysis, Wilson disease, epilepsy, Pompe disease, amyotrophic lateral sclerosis (ALS), Tay-Sachs disease, hyperoxaluria 9PH-1), spinocerebellar ataxia type 1 (SCA-1), SCA-3, u-dystrophin, Gaucher’s types II or III, arrhythmogenic right ventricular cardiomyopathy (ARVC), Fabry disease, familial Mediterranean fever (FMF), proprionic acidemia, fragile X syndrome, Rett syndrome, Niemann-Pick disease and Krabbe disease. Examples of therapeutic gene products to be expressed include N-acetylglucosaminidase, alpha (NaGLU), Treg167, Treg289, EPO, IGF, IFN, GDNF, FOXP3, Factor VIII, Factor IX and insulin.

Alternatively, or in addition as another gene product, the nucleotide sequence comprising the transgene as defined herein above may further comprise a nucleotide sequence encoding a polypeptide that serves as a selection marker protein to assess cell transformation and expression. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel, supra. Furthermore, the nucleotide sequence comprising the transgene as defined herein above may comprise a further nucleotide sequence encoding a polypeptide that may serve as a fail-safe mechanism that allows to cure a subject from cells transduced with the recombinant parvoviral (rAAV) vector of the invention, if deemed necessary. Such a nucleotide sequence, often referred to as a suicide gene, encodes a protein that is capable of converting a prodrug into a toxic substance that is capable of killing the transgenic cells in which the protein is expressed. Suitable examples of such suicide genes include e.g. the E.coli cytosine deaminase gene or one of the thymidine kinase genes from Herpes Simplex Virus, Cytomegalovirus and Varicella-Zoster virus, in which case ganciclovir may be used as prodrug to kill the transgenic cells in the subject (see e.g. Clair et al., 1987, Antimicrob. Agents Chemother. 31: 844-849).

The various modifications of the nucleotide sequences as defined herein, including e.g. the wild-type parvoviral sequences, for proper expression in insect cells is achieved by application of well-known genetic engineering techniques such as described e.g. in Sambrook and Russell (2001) “Molecular Cloning: A Laboratory Manual (3rd edition), Cold Spring Harbor Laboratory, Cold Spring Harbor Laboratory Press, New York. Various further modifications of coding regions are known to the skilled artisan which could increase yield of the encode proteins. These modifications are within the scope of the present invention.

### Cell

A cell according to the invention can be any cell that is suitable for the production of heterologous proteins. Preferably, the cell is an insect cell, more preferably, an insect cell that allows for replication of baculoviral vectors and can be maintained in culture. More preferably the insect cell also allows for replication of recombinant parvoviral vectors, including rAAV vectors. For example, the cell line used can be from Spodoptera frugiperda, Drosophila cell lines, or mosquito cell lines, e.g., Aedes albopictus derived cell lines. Preferred insect cells or cell lines are cells from the insect species which are susceptible to baculovirus infection, including e.g. S2 (CRL-1963, ATCC), Se301, SeIZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (US 6,103,526; Protein Sciences Corp., CT, USA). A preferred insect cell according to the invention is an insect cell for production of recombinant parvoviral vectors.

One of ordinary skill in the art knows how to stably introduce a nucleotide sequence into the insect genome and how to identify a cell having such a nucleotide sequence in the genome. The incorporation into the genome may be aided by, for example, the use of a vector comprising nucleotide sequences highly homologous to regions of the insect genome. The use of specific sequences, such as transposons, is another way to introduce a nucleotide sequence into a genome. The incorporation into the genome may be through one or more than one steps. Reference to the term “integrated” will be known to one in the art to also mean “stably integrated”.

In one embodiment there is provided a cell according to the invention, wherein at least one of the first and second nucleic acid construct is stably integrated in the genome of the cell. In one embodiment, the first nucleic acid construct is stably integrated in the genome of the cell. In an alternative embodiment, the second nucleic acid construct is stably integrated in the genome of the cell. In still a further embodiment, the first and second nucleic acid constructs are stably integrated in the genome of the cell.

Growing conditions for insect cells in culture, and production of heterologous products in insect cells in culture are well-known in the art and described e.g. in the above cited references on molecular engineering of insect cells (see also WO2007/046703).

An “insect cell-compatible vector” or “vector” is understood to be a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. In a preferred embodiment, the vector is a baculovirus, a viral vector, or a plasmid. In a more preferred embodiment, the vector is a baculovirus, i.e. the nucleic acid construct is a baculovirus-expression vector. Baculovirus-expression vectors and methods for their use are described for example in Summers and Smith. 1986. A Manual of Methods for Baculovirus Vectors and Insect Culture Procedures, Texas Agricultural Experimental Station Bull. No. 7555, College Station, Tex.; Luckow. 1991. In Prokop et al., Cloning and Expression of Heterologous Genes in Insect Cells with Baculovirus Vectors’ Recombinant DNA Technology and Applications, 97-152; King, L. A. and R. D. Possee, 1992, The baculovirus expression system, Chapman and Hall, United Kingdom; O′Reilly, D. R., L. K. Miller, V. A. Luckow, 1992, Baculovirus Expression Vectors: A Laboratory Manual, New York; W. H. Freeman and Richardson, C. D., 1995, Baculovirus Expression Protocols, Methods in Molecular Biology, volume 39; US 4,745,051; US2003148506; and WO 03/074714.

The number of nucleic acid constructs employed in the insect cell for the production of the recombinant parvoviral (rAAV) vector is not limiting in the invention. However, in a preferred embodiment no more than two nucleic acid constructs are employed in the insect cell for the production of the recombinant parvoviral (rAAV) vector. Preferably the two nucleic acid constructs are the first and second nucleic acids constructs as herein defined above. Preferably, the first nucleic acid construct is a Rep-Cap construct, which thus preferably comprises the first, second and third expression cassettes, whereby first and second expression cassettes resp. encode the Rep 78/68 proteins and the Rep 52/40 proteins, and the third expression cassette encodes the Cap proteins. The second nucleic acid construct is a Trans construct or a Cap-Trans construct and thus at least comprises the nucleotide sequence comprising a transgene that is flanked by at least one parvoviral inverted terminal repeat sequence.

In a preferred (DuoDuoBac) embodiment however, the second nucleic acid construct preferably also comprises an expression cassette for the Cap proteins, i.e. the fourth expression cassette. In a preferred DouDuoBac embodiment, the first nucleic acid construct comprises: i) a first expression cassette comprising a dEI promoter operably linked to the nucleotide sequence encoding the at least one of parvoviral Rep 78 and 68 proteins; ii) a second expression cassette comprising a poIH promoter operably linked to a nucleotide sequence encoding the at least one of parvoviral Rep 52 and 40 proteins; and iii) a third expression cassette comprising a poIH promoter operably linked to a nucleotide sequence encoding parvoviral VP1, VP2, and VP3 capsid proteins, preferably encoding the AAV5 VP1, VP2, and VP3 capsid proteins, whereby more preferably the VP1 initiation codon is ACG. The second nucleic acid construct comprises the transgene that is flanked by parvoviral inverted terminal repeat sequences and further the fourth expression cassette comprising a poIH promoter operably linked to a nucleotide sequence encoding parvoviral VP1, VP2, and VP3 capsid proteins, preferably encoding the AAV5 VP1, VP2, and VP3 capsid proteins, whereby more preferably the VP1 initiation codon is ACG. In this embodiment the fourth expression cassette is thus preferably identical to the third expression cassette. Preferably in this embodiment, the second and first nucleic acid constructs are present in and/or transfected into the cell in a molar ratio in the range of 5:1 to 1:10, preferably, in a molar ratio in the range of 1:1 to 1:8, more preferably in the range of 1:2 to 1:6 and most preferably in the range of 1:3 to 1:5. For example, the first nucleic acid construct can be DuoBac CapRep6 (SEQ ID NO. 10) and the second nucleic acid construct can be DuoBac CapTrans1 (SEQ ID NO. 12), wherein preferably the first and second constructs are present in a 3 : 1 molar ratio. Thereby it is understood that the “Trans” in the second construct can be any gene of interest in between the two ITRs.

A nucleotide sequence encoding parvoviral Rep proteins, is herein understood as a nucleotide sequence encoding the non-structural Rep proteins that are required and sufficient for parvoviral vector production in insect cells such the Rep78 or Rep68, and/or the Rep52 or Rep40 proteins. The parvovirus nucleotide sequence preferably is from a dependovirus, more preferably from a human or simian adeno-associated virus (AAV) and most preferably from an AAV which normally infects humans (e.g., serotypes 1, 2, 3A, 3B, 4, 5, 6, 8 and 9) or primates (e.g., serotypes 1 and 4). An example of a nucleotide sequence encoding parvovirus Rep proteins is given in SEQ ID NO. 33, which depicts a part of the AAV serotype-2 sequence genome encoding the Rep proteins. The Rep78 coding sequence comprises nucleotides 11 - 1876 and the Rep52 coding sequence comprises nucleotides 683 - 1876, also depicted separately in SEQ ID NOs. 33 and 19. It is understood that the exact molecular weights of the Rep78 and Rep52 proteins, as well as the exact positions of the translation initiation codons may differ between different parvoviruses. However, the skilled person will know how to identify the corresponding position in nucleotide sequence from other parvoviruses than AAV-2.

Preferably a nucleic acid construct of the invention, is an insect cell-compatible vector. An “insect cell-compatible vector” or “vector” is understood to be sufficient for parvoviral vector production in insect cells such the Rep78 or Rep68, and/or the Rep52 or Rep40 proteins. The parvovirus nucleotide sequence preferably is from a dependovirus, more preferably from a human or simian adeno-associated virus (AAV) and most preferably from an AAV which normally infects humans (e.g., serotypes 1, 2, 3A, 3B, 4, 5, and 6) or primates (e.g., serotypes 1 and 4). An example of a nucleotide sequence encoding parvovirus Rep proteins is given in SEQ ID NO. 33 and 19.

Therefore, in an alternative embodiment, the cell is an insect cell, and wherein at least one the first and second nucleic acid construct is an insect cell-compatible vector, preferably a baculoviral vector, and at least one expression cassette comprises at least one baculovirus enhancer element and/or at least one ecdysone responsive element, wherein preferable the enhancer element is selected from the group consisting of hr1, hr2, hr2.09, hr3, hr4, hr4b and hr5. In a preferred embodiment, the invention relates to an insect cell that comprises no more than one type of nucleotide sequence comprising a single open reading frame encoding a parvoviral Rep protein. Preferably the single open reading frame encodes one or more of the parvoviral Rep proteins, more preferably the open reading frame encodes all of the parvoviral Rep proteins, most preferably the open reading frame encodes the full-length Rep 78 protein from which preferably at least both Rep 52 and Rep 78 proteins may be expressed in the insect cell. It is understood herein that the insect cell may comprise more than one copy of the single type of nucleotide sequence, e.g. in a multicopy episomal vector, but that these are multiple copies of essentially one and the same nucleic acid molecule, or at least nucleic acid molecules that encode one and the same Rep amino acid sequence, e.g. nucleic acid molecules that only differ between each other due to the degeneracy of the genetic code. The presence of only a single type of nucleic acid molecule encoding the parvoviral Rep proteins avoids recombination between homologous sequences as may be present in different types of vectors comprising Rep sequences, which may give rise to defective Rep expression constructs that affect (stability of) parvoviral production levels in insect cells.

### Method

In a further aspect, the invention provides for a method for producing a recombinant parvoviral virion in a cell comprising the steps of:
a) culturing a cell as defined herein under conditions such that recombinant parvoviral virion is produced; and,
b) recovery of the recombinant parvoviral virion.

Recovery preferably comprises the step of affinity-purification of the virions comprising the recombinant parvoviral (rAAV) vector using an anti-AAV antibody, preferably an immobilised antibody. The anti-AAV antibody preferably is a monoclonal antibody. A particularly suitable antibody is a single chain camelid antibody or a fragment thereof as e.g. obtainable from camels or llamas (see e.g. Muyldermans, 2001, Biotechnol. 74: 277-302). The antibody for affinity-purification of rAAV preferably is an antibody that specifically binds an epitope on an AAV capsid protein, whereby preferably the epitope is an epitope that is present on capsid protein of more than one AAV serotype. E.g. the antibody may be raised or selected on the basis of specific binding to AAV2 capsid but at the same time also it may also specifically bind to AAV1, AAV3 and AAV5 capsids.

In an embodiment, the cell is an insect cell and/or wherein the parvoviral virion is an AAV virion.

In a further embodiment, wherein recovery of the recombinant parvoviral virion in step b) comprises at least one of affinity-purification of the virion using an immobilised anti-parvoviral antibody, preferably a single chain camelid antibody or a fragment thereof, or filtration over a filter having a nominal pore size of 30 - 70 nm.

Therefore, in one embodiment the invention provides a method for producing a recombinant parvoviral virion in a cell comprising the steps of:
a) culturing a cell as defined herein under conditions such that recombinant parvoviral virion is produced; and,
b) recovery of the recombinant parvoviral virion
wherein recovery of the recombinant parvoviral virion in step b) comprises at least one of affinity-purification of the virion using an immobilised anti-parvoviral antibody, preferably a single chain camelid antibody or a fragment thereof, or filtration over a filter having a nominal pore size of 30 -70 nm.

In a further aspect the invention relates to a batch of parvoviral virions produced in the above described methods of the invention. A “batch of parvoviral virions” is herein defined as all parvoviral virions that are produced in the same round of production, optionally per container of insect cells. In a preferred embodiment, the batch of parvoviral virions of the invention comprises a full virion:total virion ratio as described above and/or a full virion:empty ratio as described above.

### Constructs & Kits

In a further aspect, the invention provides for a first nucleic acid construct as defined herein.

In one embodiment, there is provided a second nucleic acid construct as defined herein.

In a further aspect, the invention provides for a kit of parts comprising at least a first nucleic acid construct as defined herein and a second nucleic acid construct as defined herein. The kit may further comprise insect cells and/or the nucleotide sequences as defined herein and/or s a nucleic acid sequence encoding baculovirus helper functions for expression in the insect cell.

### Advantages of the Invention

The inventors of the current invention have further optimised the inducible plasmid vector (expressing the parvoviral Replicase proteins) design in two ways.

Firstly, by investigating the use of alternative baculovirus promoters in regulating AAV gene expression. So far, the polyhedron promoter (poIH) has been the most extensively studied promoter in AAV production, in the BEV setting (van Oers, M. M., et al., J Gen Virol. 2015 Jan;96(Pt 1):6-23). Although alternative late promoters, such as p10, have been reported to share a host factor with poIH (Ghosh, S., et al., J Virol. 1998 Sep;72(9):7484-93), other baculovirus promoters have been reported to exhibit different induction intensities and temporal profiles (Dong, Z. Q. et al., J Biol Eng. 2018 Dec4;12:30; Lin, C. H & Jarvis, D. L., J Biotechnol. 2013 May 10;165(1):11-7; Martinez-Solis, M., et al., PeerJ. 2016 Jun 28;4:e2183). Nevertheless, their potential use for AAV production in insect cells has never been reported thus far.

Secondly, tighter regulation on the expression of AAV Rep, which is very toxic for the host cells, is also explored in this study. The use of baculovirus homologous region (hr) 2 or hr2.09 enhancer sequence in combination with poIH has become the default molecular design for the inducible OneBac platform (Aslanidi, G., et al., Proc Natl Acad Sci U S A. 2009 Mar 31;106(13):5059-64) . Here, we examined the potential use of alternative baculovirus promoters in combination with other baculovirus hr’s for the purpose of upgrading the OneBac platform, especially the OneBac Cap Trans. By studying the different baculovirus promoters and enhancers, also in different molecular conformations, we aim to optimize expression of AAV genes (Cap, Rep) which can ultimately bring a stable and robust AAV production platform yielding high quality AAV batches with high titer.

The invention thus provides for the use of alternative and non-conservative baculovirus promoters (p10, 39k, p6.9, pSel120) with similar or distinct expression intensities and temporal profiles to create inducible expression construct regulating wild-type (wt) single- or split-cassette AAV Rep, or other AAV gene expression. This enables the production of an inducible plasmid vector construct with the advantage that it is less prone to cis:trans promoter competition upon recombinant baculovirus transactivation. In addition, the novel non-hr2-0.9 baculovirus hr enhancers provide by the invention are less leaky under non-induced conditions and thereby provide the advantage of tighter regulation of the toxic Rep proteins from the inducible plasmid vector construct.

Additional benefits of the invention include an improved AAV production yield and quality over the OneBac and insect cell platform; production of an inducible promoter with no expression of toxic AAV genes, such as Rep, when switched ‘off’, which allows more viable and stable AAV packaging cells; and the adaptation of split-cassette Rep AAV design into inducible plasmid vectors.

### DESCRIPTION OF THE FIGURES

FIG. 1: In a TripleBac AAV production three baculoviruses comprising Rep, Cap and Transgene cassettes are co-infected in expresSF+ insect cells. In contrast, in the DuoBac process the Cap and Rep cassettes are combined on one baculovirus genome and co-infected into expresSF+ insect cells with a separate baculovirus containing a transgene cassette. In the DuoDuoBac production process the Cap-Rep and Cap-Trans expression cassettes are combined on two baculoviruses and co-infected in expresSF+ cells.

FIG. 2: Schematic overview of the expression cassettes and orientations of the Cap-Rep and Cap-Trans DuoBac baculovirus constructs used in the examples as well as the used single expression cassette baculoviruses.

FIG. 3: Viral titers as measured in the CLB of BacCap2 or BacCap3 DuoBac AAV productions. The productions were performed at a volumetric ratio of 5% Cap-Rep baculovirus stock and 1% transgene stock. High titers were obtained with construct DuoBac CapRep2, 3, 4 and 7 whereas low titers were obtained from DuoBac CapRep1 and 6.

FIG. 4: Total/full ratio of wtAAV5 and AAV2/5 DuoBac productions. Low total/full ratio’s (<2) are observed in AAVs produced from all DuoBac constructs. These total full ratios are significantly lower than normally observed in TripleBac AAV productions (>5 total/full, Table 2).

FIG. 5: SDS Page gel run with purified AAV material made with DuoBac CapRep 1-5. Construct DuoBac CapRep6 was not included because of low yield. DuoBac CapRep3 and DuoBac CapRep7 display correct capsid stoichiometry of 1:1:10, while DuoBac CapRep2, 4 and 5 display suboptimal capsid stoichiometry (low VP1 for DuoBac CapRep 2, 4, 5 or very high VP1 in case of DuoBac CapRep1).

FIG. 6: Gc/ip of AAVs produced with DuoBac constructs DuoBac CapRep1-6. Infectivity of produced AAVs mirrors the VP123 capsid stoichiometry of the DuoBac constructs. Here low VP1 results in low infectivity (high gc/ip) for DuoBac CapRep2, 4 and 5, while high or normal VP1 results in high infectivity (low gc/ip) for DuoBac CapRep3 and 1.

FIG. 7: SDS Page gel run with purified AAV material made with DuoBac and TripleBac production processes. The ideal capsid VP1, 2, 3 protein stoichiometry of 1:1:10 for AAV was maintained after switching to the DuoBac process (lanes 1-2, 11, 13 vs Lanes 5 - 10, 12, 14).

FIG. 8: Comparison of the total/full ratio between the DuoBac and TripleBac AAV productions.

FIG. 9: SDS Page gel run with purified DuoDuoBac and TripleBac produced AAVs. When comparing AAV made with DuoDuoBac and TripleBac process, a similar VP123 stoichiometry of 1:1:10 was observed.

FIG. 10: Formaldehyde gel run with genomic AAV DNA obtained from AAVs produced with a DuoDuoBac or TripleBac production process. AAVs produced with different Rep:Cap ratio’s using DuoDuoBac have similar genomic DNA packaged into the AAV particle. The DuoDuoBac AAV fragments match the DNA fragments found after a TripleBac production. The main band was 2.4 kb long and represents a single copy of the transgene.

### EXAMPLES

In the examples presented the inventors aim to examine the effects of using double expression cassettes (e.g. Bac.Cap-Rep with Bac.Cap-Trans or Bac.Cap-Rep with Bac.Trans) on product quality and vector yield. In Example 1 the inventors characterize the effect of the molecular optimization of double Rep-Cap cassettes on wtAAV5 and AAV2/5 yield and product quality. In example 2, the inventors produce wtAAV5 with an optimized wtAAV5 Cap-Rep and transgene baculovirus (DuoBac) and compare it against wtAAV5 produced with a triple infection. In example 3 the inventors extrapolate the DuoBac yields to larger production scale versus the Triple Bac system. Lastly, in example 4 the inventors examine the effect of using various combinations of Cap-Trans and Cap-Rep double baculoviruses (DuoDuoBac) on the quality and vector yields and compare these to triple infection wtAAV5 productions.

### Methods and Materials

### Expression Cassettes

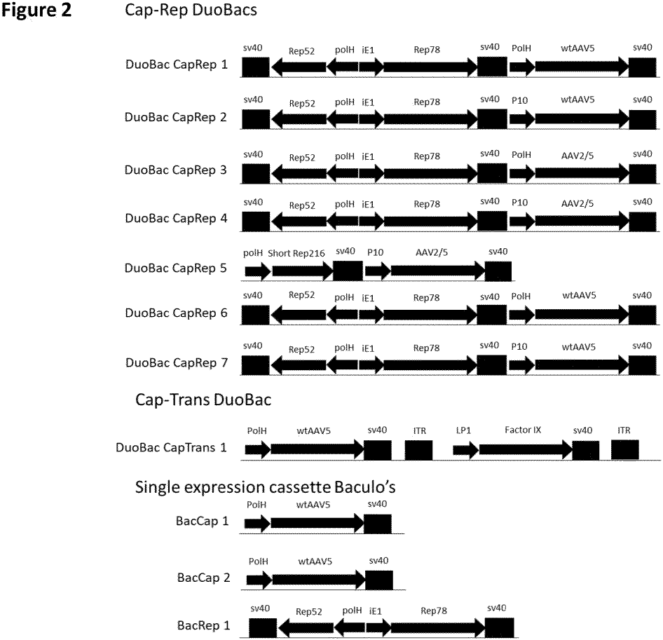

In brief, Cap-Rep DuoBac constructs (DuoBac CapRep 1 - 7) comprise a combination of a Cap cassettes (wtAAV5 or AAV2/5) under control of a Polyhedrin (PoIH) or P10 promoter and a Rep cassette. Here the Rep cassette is of split design with Rep52 and Rep78 controlled by a PoIH and dIE1 promoter, respectively. DuoBac CapTrans1 combines a wtAAV5 Cap cassette under control of the PoIH promoter with a BacTrans4 transgene cassette. Single expression cassette constructs were needed as well, both for DuoBac and TripleBac AAV productions. These constructs were always kept the same and are BacCap1 or BacCap2, (wtAAV5) and BacRep1, split-.Rep cassette. FIG. 2 summarizes the orientations used in the cassette designs, while Tables 1A and 1B summarize the different promoter/start codon combinations used per construct.

TABLE 1A

Cap-Rep DuoBac promoter/start codon combinations per construct

| | | | |
|---|---|---|---|
| construct | (promoter-start codon) Rep52 | (promoter-start codon) Rep78 | promoter-Vp1 start-codon) Cap |
| DuoBac CapRep1 | PoIH-ATG-Rep52 | dIE1-ATG-Rep78 | PoIH-CTG-wtAAV5 |
| DuoBac CapRep2 | PoIH-ATG-Rep52 | dIE1-ATG-Rep78 | P10-CTG-wtAAV5 |
| DuoBac CapRep3 | PoIH-ATG-Rep52 | dIE1-ATG-Rep78 | PoIH-ACG-AAV2/5 |
| DuoBac CapRep4 | PoIH-ATG-Rep52 | dIE1-ATG-Rep78 | P10-ACG-AAV2/5 |
| DuoBac CapRep5 | PoIH-ACG-ShortRep | - | P10-ACG-AAV2/5 |
| DuoBac CapRep6 | PoIH-ATG-Rep52 | dIE1-ATG-Rep78 | PoIH-ACG-wtAAV5 |
| DuoBac CapRep7 | PoIH-ATG-Rep52 | dIE1-ATG-Rep78 | P10-DoubleATG-wtAAV5 |
| BacRep1 | PoIH-ATG-Rep52 | dIE1-ATG-Rep78 | - |
| BacCap1 | - | - | PoIH-CTG-wtAAV5 |
| BacCap2 | - | - | PoIH-ACG-wtAAV5 |

TABLE 1B

Cap-Trans DuoBac transgene and Cap promoter/start codon combination

| | | |
|---|---|---|
| Construct | Transgene | (promoter-VP1 startcodon) Cap |
| DuoBac CapTrans 1 | BacTrans 4 | PoIH-ACG-wtAAV5 |

### Cell Culture and Baculovirus Amplification

ExpresSF+ insect cells were maintained in SF-900II SFM medium (Gibco) in shaker flasks at 28° C. at 135 RPM. Fresh baculovirus was generated for the productions of each example. Here ExpresSF+ cells were inoculated with frozen baculovirus stocks at a concentration of 3 ul stock /ml insect cells. 72 hours after the start of infection fresh baculovirus was harvested by centrifuging the cells at 1900 xg for 15 minutes and storing the cell supernatant.

### Production and Purification of AAV

AAV material was generated by volumetrically co-infecting expresSF+ insect cells with various combinations of freshly amplified recombinant baculoviruses comprising double expression cassettes (Cap-Rep and Cap-Trans) or single expression cassettes (Cap, Rep, Trans) or a combination of double expression (Cap-Rep) and single (Trans) expression cassettes. The exact ratios are described in the examples. Following a 72 hour incubation at 28° C., cells were lysed in lysis buffer (1.5 M NaCl, 0.5 M Tris-HCl, 1 mM MgCl₂, 1% Triton x-100, pH=8.5) for 1 hour. Next, genomic DNA was digested with benzonase (Merck) at 37° C. for 1 hour after which cell debris was pelleted at 1900 xg for 15 minutes (crude lysate samples). Supernatant was stored at 4° C. until the start of purification. AAV was then purified from crude lysed bulk (CLB) by batch binding with AVB Sepharose (GE healthcare). In brief, AVB sepharose resin was washed in 0.2 M HPO₄ pH=7.5 buffer, after which clarified crude lysate was added to the resin and incubated 2 hours at room temperature (RT) in an incubator shaking at 85 rpm. Resin was washed again in 0.2 M HPO₄ pH=7.5 buffer. Next, bound virus was eluted from the resin with the addition of 0.2 M Glycine pH=2.5. The pH of the eluted virus was immediately neutralized by the addition of 0.5 M Tris-HCl pH=8.5 and stored at -20° C. until further use.

### Titration by Q-PCR and Total/Full Ratio Measurement by A260/A280 or HPLC

Viral titers of the crude lysates and purified AAV batches were determined by Q-PCR. Q-PCRs were run with primers specific for the promotor region of the transgene. Q-PCRs were run on an Applied Biosystems 7500 fast Q-PCR systems. Total/full ratios of purified AAV batches were measured by UV/Vis spectrophotometry. 1 ul of 10% SDS was mixed with 100 ul of purified AAV and incubated at 75° C. for 10 minutes. Following heat treatment, the absorbance at 260 and 280 nm was measured on a Nanodrop. Using the calculation described by Sommer et al. 2003 the total/full ratio of the AAV material was calculated. Alternatively, total particles were measured by HPLC. Here purified AAV material is loaded onto a size exclusion column. Total particles are determined via integrating the area under the curve of the capsid peak. Total/full ratio is subsequently calculated by dividing the total particles with the virus titer measured by the Q-PCR.

### Total Protein Gels of Purified AAV Batches

Purified AAV batches were diluted in 4x Laemmli Sample Buffer (Biorad) supplemented with 10% β-mercaptoethanol (Bio-Rad), heated for 5 minutes at 95° C. and loaded on a 4-20% Mini-PROTEAN® TGX Stain-Free gel (Biorad). After 35 minutes of electrophoresis at 200 Volt in TGS buffer (Biorad) the gel stain was developed by exposing the gel for 5 minutes under UV light and visualizing the bands on a Chemidoc touch imager (Biorad).

### Infectivity Assay in HelaRC32

The number of genome copies required for a single infectious particle (gc/ip) was determined with a limiting dilution based infectious titer assay. In brief, HelaRC32 (ATCC) cell that stably express AAV-derived Rep and Cap proteins were transduced with a series of AAV dilutions in replicates of 10 and infected with or without WT adenovirus 5 (wtAd5) at a wtAd5:HeLaRC32 MOI of 50. Plates were incubated for 48 h at 37° C. and wells were assessed for the presence or absence of vector genome DNA by means of Q-PCR using a vector genome-specific primer probe set. The number of infectious particles per seeded vector genome was calculated according to the Spearman-Kärber method [5].

### Formaldehyde Gel Electrophoresis With Genomic AAV DNA

Genomic AAV DNA was isolated from purified AAV batches with the PCR purification Nucleospin kit (Machery Nagel). Prior to the electrophoresis run 500 ng of AAV genomic DNA was denatured for 10 minutes at 95° C. in formaldehyde loading buffer (1 ml 20x MOPS, 3.6 ml 37% Formaldehyde, 2 ml 5 mg/ml Orange G in 67% sucrose, to 10 ml with MQ) and immediately put on ice. Next, samples were run on a 1% agarose gel made in 1x MOPS (40 mM MOPS, 10 mM NaAc, 1mM EDTA, pH=8.0) supplemented with 6.6% formaldehyde. Samples were then run for 2 hours at 100 volts in 1x MOPS supplemented with 6.6% formaldehyde running buffer. After the run, DNA was stained with SYBR Gold (Thermofisher) and bands were visualized on a Chemidoc touch imager (Biorad).

### Design of Experiments (DoE) Methodology

To study the effects of upstream bioprocess variance on the total:full ratios of the DuoBac and TripleBac systems two studies were subjected to Design of experiments (DoE) methodology and analysis. The two studies were performed using slightly different methods, however in both cases experimental variance was introduced in shaker flasks and AAV purification was performed using comparable methods. In addition, for both studies, two types of analysis were performed on purified samples for each experimental condition: qPCR was used to determine the vector genome copy number (gc), while SEC-HPLC was used to determine the total amount of particles regardless of content. These two metrics were subsequently used to calculate the total:full ratio, representing the proportion of total AAV capsids relative to full capsids containing a genome copy. The differences between both studies are described in the two subsequent sections.

### DoE DuoBac System: Design Space and Experimental Platform

By means of a Central Composite Design (CCD), experimental variance was introduced during DuoBac-mediated transduction of Sf+ cells as listed in Table_2. This yielded a total of 17 experimental conditions (“production cultures”) with three replicate mid-points.

TABLE 2

Design space for the DuoBac transduction system

| Factor | Low | Mid | High |
|---|---|---|---|
| BacTrans5 (% vol.) | 0.33 | 1 | 3 |
| DuoBac CapRep3 (% vol.) | 0.33 | 1 | 3 |
| VCD at TOI (x10⁶ VC/mL) | 1 | 1.45 | 1.9 |

Amplified baculovirus and seed cells were generated in 10 L wave bags (Flexsafe, Sartorius) using rocking motion bioreactors (BioWave PU-Biostat, Sartorius). The media used throughout this study was Sf900 II media (ThermoFisher). The settings for all incubations were as follows T=28° C.; agitation at 25 rpm and 8 ° angle; DO=50%; and an airflow rate of 0.2 L/min. One dedicated bioreactor was used for amplification of cells at a working volume of 5 L and an initial VCD at 1.2 x 10⁶ VC/mL (reactor A). 18.5 hours after inoculating reactor A, two bioreactors were inoculated at a concentration of 0.8 x 10⁶ VC/mL and a working volume of 5.25 L (reactors B and C). 15.75 mL baculovirus Working Seed Virus (WSV) was added to reactors B and C 18 hours after cell inoculation for separate amplification of baculoviruses BacTrans5 and DuoBac CapRep3. After an additional 48 hours of incubation all reactors were harvested. The resulting materials (cells and baculovirus) were used to prepare AAV production cultures.

For production cultures, a fresh media-exchange step was implemented prior to transduction to control VCD at TOI and media composition. This media exchange involved gentle centrifugation of each seed culture at 300 g, discarding the supernatant and resuspending cells in fresh media to achieve a target VCD at TOI. Production culture composition was done as specified in Table 2.

After 70 hours, the transduction was terminated by consecutive steps of lysis (addition of 10% v/vof a 10x lysis buffer, incubation for 60 minutes at 37° C. and 135 rpm), benzonase treatment (addition of 10 units Benzonase per mL, incubation for 60 minutes at 37° C. and 135 rpm), clarification (centrifugation for 15 minutes at 4100 g at RT) and filtration (filtration through a 0.22 µm bottle top filter under a vacuum). The filtrates were incubated at RT for 12 hours for adventitious viral inactivation. Remaining filtrates were purified using a batch binding affinity chromatography protocol which involved (1) preparation of AVB Sepharose HP resin in 0.2M phosphate buffer pH 7.5 (1:1 volumetric ratio); (2) addition and incubation of 250 µL resin suspension to 40 mL of filtrate for 4 hours at 40 rpm; (3) centrifugation of resin at 4100 g for 5 minutes; (4) washing pellets with 0.2 M phosphate buffer pH 7.5; (5) extracting the pellet using 500 µL 0.5 M Glycine/HCl pH 2.5 during an incubation of 4 minutes; (6) centrifuging the used pellet using a benchtop centrifuge; (7) neutralizing the supernatant using 200 µL Tris/HCl pH8.5 buffer; and (8) filtering the neutralized eluate with a 0.22 µm PVDF syringe filter. The purified materials were used for qPCR and SEC-HPLC analysis to determine total:full ratios.

### Results

### Example 1: Characterization of wtAAV5 and AAV2/5 Cap-Rep DuoBac Constructs

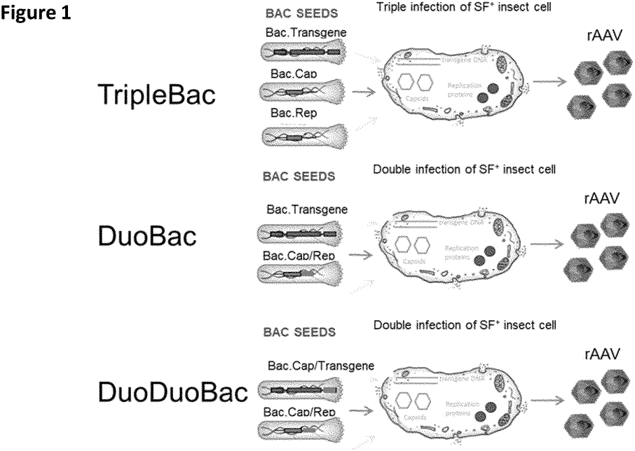

AAV production in insect cells is commonly performed by co-infecting three baculoviruses comprising Rep, Cap and Trans cassettes. To improve the statistical chance that all three components are present in the cell at the same time the Cap and Rep expression cassettes were moved to a single baculovirus (FIG. 1). To investigate if the quality and quantity of wtAAV5 and AAV2/5 produced in a double infection setting may be improved, the inventors swapped the single Rep expression cassette for a split Rep expression cassette and optimized the promoter/VP1 start codon combination of Cap. Introduction of the split Rep cassette can give better control over the timing and expression strength of Rep52 and Rep78. Furthermore, optimization of the VP123 ratio of the capsids is essential for generating infective AAV.

Constructs DuoBac CapRep1-7 (Table 1A and FIG. 1) were designed to optimize the expression of wtAAV5 and AAV2/5 Cap and balance them with Rep expressed from a split Rep cassette. To assess the impact of these changes on the AAV vector yields and quality, DuoBac productions were performed with a therapeutically relevant transgene (BacTrans4). AAVs were produced in expresSF+ insect cells (50 ml) with 5% freshly amplified Cap-Rep baculovirus and 1% freshly amplified transgene baculovirus. Following the production, viruses were purified and several assays were performed on the resulting AAV material. Virus titers (by Q-PCR) were determined on the crude lysates. Total/full ratio’s (by HPLC/Q-PCR) and capsid stoichiometry (by SDS-page gel) were determined on purified AAVs. The number of genome copies required for 1 infectious particle (gc/IP) was determined with an infectivity assay in HelaRC32 cells.

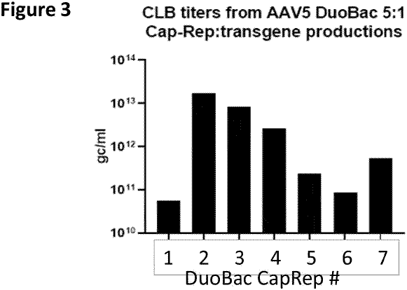

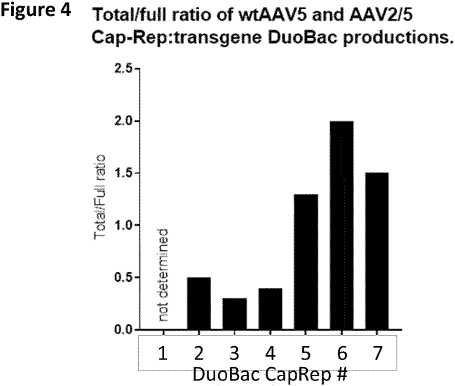

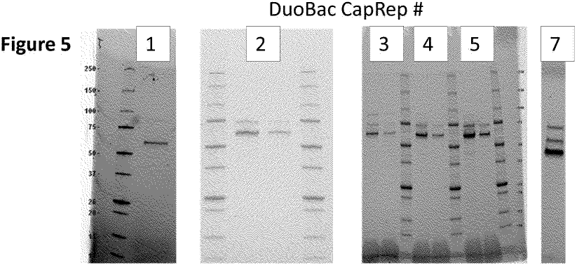

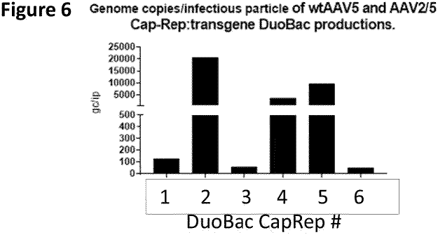

FIG. 3 summarises the viral titers measured in crude lysates of the wtAAV5 and AAV2/5 DuoBac productions. High viral yields (>1e11 gc/ml) were obtained with constructs DuoBac CapRep2, 5 and 7, while relatively low yields were observed with constructs DuoBac CapRep1 and 6. The total/full ratio of the purified virus batches was determined by dividing the total particles/ml (as determined by HPLC) by the genome copies/ml (as determined by Q-PCR). In general, low total/full ratio’s (<2.0) were observed with all DuoBac constructs (FIG. 4). This observation contrasts significantly with the total/full ratio normally observed in TripleBac AAV productions which normally falls above 5 (see example 2). Capsid stoichiometry of the purified AAVs was determined by SDS-Page gel electrophoresis (FIG. 5, capsid stoichiometry of DuoBac CapRep6 could not be determined due to low virus yield). Capsid stoichiometry was significantly impacted depending on which DuoBac construct was used. DuoBac CapRep3 and 7 display correct capsid stoichiometry of 1:1:10, while DuoBac CapRep2, 4 and 5 display suboptimal capsid stoichiometry (low VP1 for DuoBac CapRep2, 4 and 5 or very high VP1 in the case of DuoBac CapRep1). The effect that these changes could have on AAV infectivity was determined by a limiting dilution infectivity assay in HelaRC32 (FIG. 6). AAV infectivity results mirrored the capsid stoichiometry results. Here DuoBac CapRep1, 3 and 6 showed high infectivity (low gc/ip), due to normal or high VP1 in the capsid. While DuoBac CapRep2, 4 and 5 (high gc/ip) showed reduced infectivity due to a low amount of VP1 in the Capsid. Table 3 summarizes the data from these experiments.

TABLE 3

Summary of the quality parameters of AAV produced with DuoBac constructs DuoBac CapRep1-7

| construct | (promoter-startcodon) Rep52 | (promoter-startcodon) Rep78 | (promoter-Vp1 startcodon) Cap | gc/ml in CLB | Total-full ratio | VP123 ratio | gc/ip |
|---|---|---|---|---|---|---|---|
| DuoBac CapRep1 | PoIH-ATG-Rep52 | dIE1-ATG-Rep52 | PoIH-CTG-wtAAV5 | 5.57 E+10 | n/d | High VP1 | 129 |
| DuoBac CapRep2 | PoIH-ATG-Rep52 | dIE1-ATG-Rep52 | P10-CTG-wtAAV5 | 1.76 E+13 | 5,0 | low VP1 | 20825 |
| DuoBac CapRep3 | PoIH-ATG-Rep52 | dIE1-ATG-Rep52 | PoIH-ACG-AAV2/5 | 8.45 E+12 | 0,3 | normal | 60 |
| DuoBac CapRep4 | PoIH-ATG-Rep52 | dIE1-ATG-Rep52 | P10 -ACG-AAV2/5 | 2.64 E+12 | 0,4 | low VP1 | 3591 |
| DuoBac CapRep5 | PoIH-ACG-ShortRep | - | P10 -ACG-AAV2/5 | 2.37 E+11 | 1,3 | low VP1 | 9651 |
| DuoBac CapRep6 | PoIH-ATG-Rep52 | dIE1-ATG-Rep52 | PoIH-ACG-wtAAV5 | 8.8 E+10 | 2 | n/d | 52,8 |
| DuoBac CapRep7 | PoIH-ATG-Rep52 | dIE1-ATG-Rep52 | P10 -doubleATG-wtAAV5 | 5.3 E+11 | 1,5 | normal | n/d |

From these results it appears that promoter competition has a significant impact on the virus titers for wtAAV5 DuoBac constructs (PoIH Rep + PoIH Cap= low titer for wtAAV5, DuoBac CapRep1 and 6), but less for AAV2/5 (PolH Rep+ PolH Cap = high titer for AAV2/5, DuoBac CapRep3). Introducing a P10 promoter before the wtAAV5 cassette improves the titer (DuoBac CapRep2), but results in a suboptimal VP123 stoichiometry. Introducing a stronger start codon in front of VP1 (double ATG) rescues VP123 stoichiometry and produces high titers (DuoBac CapRep7). This shows that balancing the promoter type and initiation strength for Cap VP1 is essential for generating high titers with correct AAV capsid stoichiometry. Furthermore, process complexity is reduced by combining Rep and Cap on the same baculovirus. This combination of AAV genes also led to clear improvements to the total/full ratio. How DuoBac AAV production compares to TripleBac AAV production will be examined in example 2.

### Example 2: Comparison of AAV5 DuoBac (Bac.Cap-Rep and Bac.Transgene) and Triple Bac (Bac.Cap, Bac.Rep Bac.Transgene) AAV Productions

The previous example showed that by combining the Cap and Rep cassette on the same baculovirus and molecularly optimizing the Cap cassette we were able to produce an improved AAV product. This example compares AAV produced by a DuoBac and TripleBac process. To compare the two production systems DuoBac (DuoBac CapRep 7: Cap wtAAV5-Rep) productions were compared to TripleBac AAV productions (BacCap1 wtAAV5, BacRep1) with respect to vector yields and quality. Both a reporter and two therapeutically relevant transgenes were used in the AAV productions (BacTrans 1, 3 and 4). To perform AAV productions, expresSF+ insect cells (50 ml or 2.5 L) were inoculated with multiple volumetric ratios of freshly amplified baculovirus stocks. Inoculation volumes ranged between 1 to 5% of the culture volume. Following production, viruses were purified and several assays were performed on the material. Virus titers (in gc/ml by Q-PCR) were determined on crude lysates and purified AAVs. Total/full ratio’s (by A260/A280) and VP123 ratio (by SDS-page gel) were determined on purified AAV material.

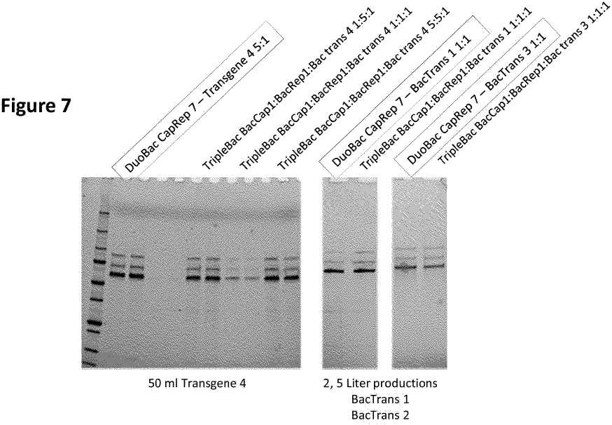

Table 4 summarizes the 50 ml production results, while Table 5 summarizes the 2.5 L production results. Both at 50 ml and 2.5 L scale, DuoBac productions outperform TripleBac productions in both virus yields and total/full ratio. Depending on the inoculation volumes or transgenes used forthe production, titers (in gc/ml) in the CLB improved by 4 to 10-fold with DuoBac CapRep 7 as compared to the equivalent TripleBac production. Total genome copies purified from the productions were increased with a similar factor. Interestingly total/full ratios were also improved with the DuoBac process. Here, the used transgene seems to influence the amount this parameter improves, but the total/full ratio was consistently improved in the DuoBac productions (approximately 2-8 fold depending on the transgene cassette used for production). Expression of VP123 capsid proteins was identical between the DuoBac and TripleBac AAV productions (FIG. 7), maintaining the ideal stoichiometry of 1:1:10.

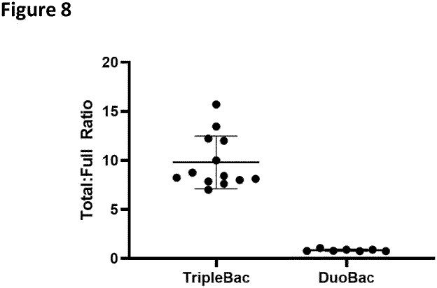

Reducing process complexity by combining the Cap and Rep expression cassettes on the same baculovirus resulted in clear improvements in yield and total/full ratio (FIG. 8), whilst maintaining the ideal VP protein stoichiometry of AAV. Although not investigated here, it is likely that process robustness (batch to batch variation) can be improved with a DuoBac process because of the reduction from three to two variables.

TABLE 4

Production results for the 50 ml DuoBac to TripleBac comparison

| Volumetric Ratio’s 50 ml productions | gc/ml crude lysate | Total gc | Total/Full Ratio |
|---|---|---|---|
| DuoBac CapRep7 : BacTrans4 5:1 | 5.30 E+11 | 2.65 E+13 | 1,5 |
| BacCap1 : BacRep1 : BacTrans4 1:1:1 | 6.10 E+10 | 3.05 E+12 | 2,4 |
| BacCap1 : BacRep1 : BacTrans4 1:5:1 | 2.70 E+10 | 1.35 E+12 | 1,4 |
| BacCap1 : BacRep1:BacTrans4 5:5:1 | 1.5 e11 | 7.50 E+12 | 2,1 |

TABLE 5

Production results for the 2.5 Liter DuoBac to TripleBac comparison

| Volumetric Ratio’s 2.5 L productions | gc/ml crude lysate | Total gc from 2.5 L | Total/Full Ratio |
|---|---|---|---|
| BacCap1 : BacRep1 : BacTrans1 1:1:1 | 8.90 E+10 | 2.25 E+14 | 6,7 |
| DuoBac CapRep7 : BacTrans1 1:1 | 1.10 E+12 | 2.80 E+15 | 1,5 |
| BacCap1 : BacRep1 : BacTrans3 1:1:1 | 2.40 E+11 | 5.90 E+14 | 16,3 |
| DuoBac CapRep7 : BacTrans3 1:1 | 6.80 E+12 | 1.70 E+16 | 1,8 |

### Example 3: Comparison of DuoDuoBac (Bac.Cap-Rep and Bac.Cap-Trans) to TripleBac AAV (Bac.Cap, Bac.Rep Bac.Transqene)

Previous studies showed that the Cap:Rep baculovirus inoculation ratio of a TripleBac AAV production had a direct impact on the total/full ratio and titer yield of an AAV production. Here increased Rep baculovirus inoculation resulted in a reduction in Capsid production and total/full ratio. In contrast, an increased Cap baculovirus inoculation ratio increased the total/full ratio and yield. By introducing a Cap cassette on both the Rep and Transgene baculoviruses, thereby creating a double DuoBac process or DuoDuoBac process (FIG. 1), we have more freedom controlling the Cap:Rep ratio in the cell during an AAV production. Also it would allow us to explore Cap:Rep production ratios (especially high Cap ratios) that are impossible to achieve in a TripleBac AAV process (due to too high inoculation volume which inhibits AAV production).

In this Example we aim to investigate the impact of changing the Cap:Rep ratios during insect cell infection on AAV quality and yield, this was achieved by varying the DuoBac CapTrans1 to DuoBac CapRep6 inoculation ratio. The DuoDuoBac AAV production was compared to TripleBac AAV productions. AAV productions were performed in expresSF+ insect cells at a 50 ml scale. Inoculation volumes ranged between 1 to 5% of the culture volume for each baculovirus. Following production, viruses were purified with AVB sepharose. Virus titers (gc/ml as determined by Q-PCR) were measured in the crude lysates and purified AAVs. Total/full ratio’s (by A260/A280) and capsid composition (by SDS-page gel) were determined on purified AAVs. In addition, the genomic DNA packaged into the AAV particle was also investigated by formaldehyde gel electrophoresis.

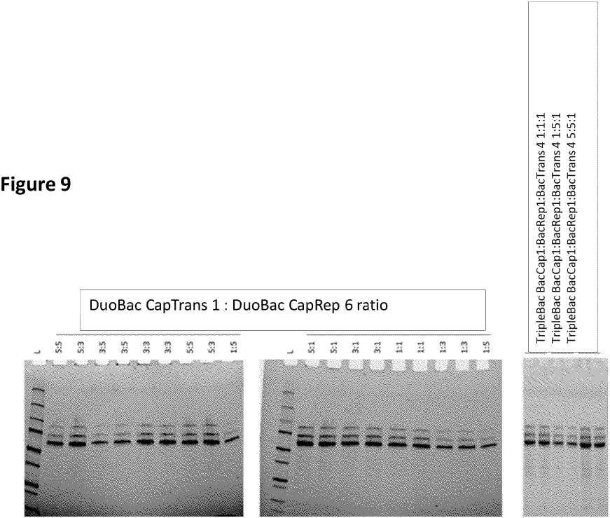

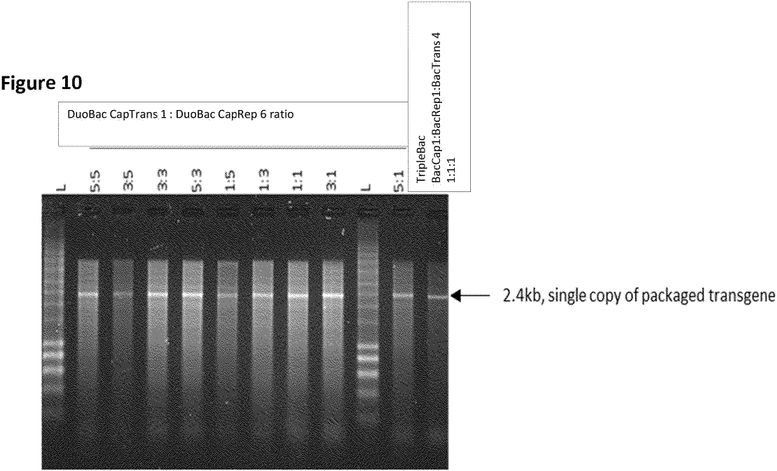

Table 6 summarises the result of the DuoDuoBac and TripleBac AAV productions. For the DuoDuoBac productions it lists the used inoculation conditions as well as what the equivalent inoculation conditions would be needed to achieve a similar ratio with a TripleBac AAV production. In all of the of the DuoDuoBac AAV productions tested, the vector yields in the crude lysate fell between 7 e+11 to 1.4 e+12 gc/ml, as compared to 6-7e+11 for the tested TripleBac productions, meaning a 2-fold titer increase is observed for the best DuoDuoBac condition. The total/full ratio of all DuoDuoBac productions was reduced as compared to TripleBac productions. When comparing DuoDuoBac productions, a lower total/full ratio was generally observed when more Rep was present, whilst a higher total/full ratio was linked to an increase in Cap. The best condition tested was the 1:3 DuoBac CapTrans1 to DuoBac CapRep6 co-infection, which resulted in an average titer in the CLB of 1.2 e+12 gc/ml with a total/full ratio of ~1.5. Compared to its closest TripleBac equivalent (5:5:1 ratio), the titer was improved by 2-fold (1.2 e+12 vs 6 e+11), while the total/full ratio was improved approximately 4-fold (1.5 vs 6). When comparing the expression of capsid proteins VP-1, -2 and -3 between DuoDuoBac and TripleBac productions, a similar stoichiometry of 1:1:10 was observed for all conditions tested (FIG. 9). This indicated that introducing the Cap cassette on the Rep and Transgene baculoviruses did not alter the optimal ratio, maintaining it at 1:1:10. Also, the genomic DNA packaged into the AAV particle was similar between DuoDuoBac and TripleBac productions (FIG. 10). Genomic AAV DNA isolated from both productions resulted in an identical banding pattern on a formaldehyde gel. The main band was 2.4 kb long and represents a single copy of the BacTrans4 transgene.

In summary, a DuoDuoBac process results in improved vector yields and total to full ratios using a wide range of Bac.Cap-Rep to Bac.Cap-Trans inoculation ratios as compared to TripleBac. Increased freedom to change the Cap:Rep ratio in the production cell during AAV production (due to the presence of two Cap expression cassettes and the reduction of the number of baculovirus seeds used for infection) allows for steering and optimisation of the total/full ratio of the produced AAVs. We observed that an increase in Rep resulted in slightly lower yields and total/full ratio, while an increase in Cap resulted in higher total/full ratio. DuoDuoBac productions minimize the variation in yield and total/full ratio as compared to TripleBac. In addition, a DuoDuoBac AAV production allows us explore Cap:Rep ratios that cannot be feasibly reached with a TripleBac process. This expanded manoeuvring room offered by the DuoDuoBac process can potentially allow for the development of more robust AAV productions processes.

TABLE 6

Production results for the 50 ml DuoDuoBac to TripleBac comparison

| Shaker Flask# | ratio DuoBac CapTrans 1: DuoBac CapRep6 | equivalent Cap:Rep:Transgene | gc/ml Crude Lysate | Total Gc | Total/full ratio |
|---|---|---|---|---|---|
| 1 | 5:5 | 10:5:5 | 9.6 E+11 | 4.80 E+13 | 2,0 |
| 2 | 5:5 | 10:5:5 | 1.1 E+12 | 5.50 E+13 | 2,0 |
| 3 | 5:3 | 8:3:5 | 1.2 E+12 | 6.00 E+13 | 2,5 |
| 4 | 5:3 | 8:3:5 | 1.3 E+12 | 6.50 E+13 | 1,9 |
| 5 | 3:5 | 8:5:3 | 9.1 E+11 | 4.55 E+13 | 2,7 |
| 6 | 3:5 | 8:5:3 | 7.3 E+11 | 3.65 E+13 | 1,1 |
| 7 | 3:3 | 6:3:3 | 1.2 E+12 | 6.00 E+13 | 1,7 |
| 8 | 3:3 | 6:3:3 | 1.3 E+12 | 6.50 E+13 | 1,9 |
| 9 | 1:5 | 6:5:1 | 8.9 E+11 | 4.45 E+13 | 1,3 |
| 10 | 1:5 | 6:5:1 | 8.2 E+11 | 4.10 E+13 | 1,7 |
| 11 | 1:3 | 4:3:1 | 1.1 E+12 | 5.50 E+13 | 1,1 |
| 12 | 1:3 | 4:3:1 | 1.1 E+12 | 5.50 E+13 | 1,8 |
| 13 | 1:1 | 2:1:1 | 1.4 E+12 | 7.00 E+13 | 2,2 |
| 14 | 1:1 | 2:1:1 | 1.3 E+12 | 6.50 E+13 | 2,5 |
| 15 | 3:1 | 4:1:1 | 1.3 E+12 | 6.50 E+13 | 3,6 |
| 16 | 3:1 | 4:1:1 | 8.8 E+11 | 4.40 E+13 | 3,2 |
| 17 | 5:1 | 6:1:1 | 8.8 E+11 | 4.40 E+13 | 4,6 |
| 18 | 5:1 | 6:1:1 | 1.1 E+12 | 5.50 E+13 | 5,0 |
| 19 | 1:1:1 Triple infection (BacCap2: BacRepl: BacTrans 4) | 1:1:1 | 6.3 E+11 | 3.15 E+13 | 7,6 |
| 20 | 1:1:1 Triple infection (BacCap2: BacRepl: BacTrans 4) | 1:1:1 | 6.0 E+11 | 3.00 E+13 | 6,9 |
| 21 | 1:5:1 Triple infection (BacCap2: BacRepl: BacTrans 4) | 1:5:1 | 5.9 E+11 | 2.95 E+13 | 2,9 |
| 22 | 1:5:1Triple infection (BacCap2: BacRepl: BacTrans 4) | 1:5:1 | 5.9 E+11 | 2.95 E+13 | 3,2 |
| 23 | 5:5:1 Triple infection (BacCap2: BacRepl: BacTrans 4) | 5:5:1 | 1.2 E+12 | 6.00 E+13 | 6,3 |
| 24 | 5:5:1 Triple infection (BacCap2: BacRepl: BacTrans 4) | 5:5:1 | 6.6 E+11 | 3.30+13 3.30 E+13 | 6,1 |

### Example 4: Comparison of DuoDuoBac (Bac.Cap-Rep and Bac.Cap-Trans) to DuoBac AAV (Bac.Cap, Bac.Rep Bac.Transqene)

### 4.1 Cell Culture and Baculovirus Amplification

ExpresSF+ insect cells were cultured in SF-900II SFM medium under conditions as described above. Fresh baculovirus inocula were generated as described above.

### 4.2 DOE Studies in 1L Shake Flasks

### 4.2.1 DOE deSign

A Central Composite Design (CCD) was used to investigate two factors (volumetric infection ratios of the two amplified baculoviruses in a range of 0.33-3%) and their interactions. Statistical analysis was performed using Design Expert 11 (Statease, Minneapolis MN) and JMP 15 (SAS Institute Inc., Cary, NC). Quadratic Response Surface Models were generated using a rotatable CCD (α=1.414) and three center points. Genome copy titers in filtered crude lysed bulk and total particle to genome copies (tp/gc) ratios were set as responses. Only statically significant model terms (p<0.1) were included in each model and were selected through stepwise regression whilst maintaining model hierarchy.

### 4.2.2 Production and Purification of AAV

Amplified baculovirus and seed cells (preculture) were generated in 1L shake flasks at 28° C. at 135 rpm. The media used throughout this study was SF900 II media (ThermoFisher). Based on the VCD of the preculture, a calculated volume of culture is added to each 1 L shake flask to achieve the target seeding cell density of 1.3 x 10⁶ VC/mL in a final working volume of 400 mL. Additional SF900 II medium was added to each shake flask to bring the culture volume to 400 mL, as needed. Cell expansion in 1 L shake flasks was performed at 28° C. and 135 rpm. 15-21 hours after inoculation, a pool of amplified baculovirus inocula was added at a volumetric infection ratio according to DOE design. After infection, temperature set-point was increased to 30° C. and the cultures were continued for 68-76 hours at 135 rpm. After that, the cultures were harvested by adding 10% (v/v) of 10x lysis buffer (Lonza). 30 minutes after starting lysis, temperature setpoint was increased to 37° C. When the temperature set-point was reached, benzonase was added (9 unites/mL), after which the culture was incubated for additional 60 minutes. Clarification of crude lysed bulk was performed by centrifugation for 15 minutes at 4100 g and room temperature (20- 25° C.) followed by filtration through a 0.2 µm membrane filter. Filtered bulks were then purified using AVB Sepharose HP resin from Cytiva. The product was eluted using 0.2 M glycine/HCI pH 2.4 buffer and subsequently neutralized using 60 mM Tris pH 8.5. Purified samples were subsequently analyzed by qPCR (to determine the vector genome copy number, GC concentration in crude lysate) and SEC-HPLC (to determine the total amount of total AAV particles). The results in Table 7 show that the DuoDuoBac system achieve higher vectors yields than a comparable DuoBac system over a wide range of infection ratios of the two baculoviruses.

TABLE 7

The effects of different volumetric infection ratios of the two baculoviruses on AAV vector yields and total to full ratios for DuoBac and DuoDuoBac produced in 1 L shake flasks

| BacIDs | Infection ratio range (%) | GC (E+11 gc/mL) | TP/GC ratio |
|---|---|---|---|
| DuoBac BacCapRep6 + BacTrans4 | 0.33-3.00 | 0.1-2.7 | 2.0 - 3.2 |
| DuoDuoBac BacCapTrans1 + BacCapRep6 | 0.33-3.00 | 1.7 - 4.3 | 3.4 - 25.3 |

### 4.3 Productions in 2 L Stirred Tank Bioreactors

### 4.3.1 Production and Purification of AAV

Amplified baculovirus and seed cells (preculture) were generated in 1 L shake flasks at 28° C. at 135 rpm. The media used throughout this study was SF900 II media (ThermoFisher). For each combination of baculoviruses, rAAV production was performed in duplicate, using two 2 L stirred tank reactors (STR, The UniVessel® SU, Satorious). Based on the VCD of the preculture, a calculated volume of culture is added to the 2 L STR to achieve the target seeding cell density of 0.5 x 10⁶ VC/mL in a final working volume of 2 L. Additional SF900 II medium was added to the 2 L STR to bring the culture volume to 2 L, as needed. Cell expansion in 2 L STR was performed at 28° C. Dissolved oxygen (DO) was maintained at 30% with a continuous fixed air flow through overlay at 0.2 L/min and oxygen addition through sparger at a flow of 0-150 ccm using a stirring speed of 100-300 rpm. 43-48 hours after inoculation, a pool of amplified baculovirus inocula was added at a volumetric infection ratio indicated in Table 8. After infection, temperature set-point was increased to 30° C. and the cultures were continued using the settings described above.

The cultures were harvested 68-76 hours post-infection by adding 10% (v/v) of 10x lysis buffer (Lonza). 30 minutes after starting lysis, temperature setpoint was increased to 37° C. When the temperature set-point was reached, benzonase was added (9 unites/mL), after which the culture was incubated for additional 60 minutes. Clarification of crude lysed bulk was performed by centrifugation for 15 minutes at 4100 g and room temperature (20-25° C.) followed by filtration through a 0.2 µm membrane filter. Filtered bulks were then purified using a column packed with AVB Sepharose HP resin from Cytiva. The product was eluted using 0.2M glycine/HCI 2 M urea pH 2.4 buffer and subsequently neutralized using 60 mM Tris 2 M urea pH 8.5. Neutralized eluate was then loaded onto 5 mL Mustang Q membrane (Pall). Product elution was performed using 60 mM Tris 150 mM NaCl 2 M urea pH 8.5 buffer, followed by a nanofiltration using Planova 35N filter (0.01 m²). Finally, product was diafiltered against phosphate buffered saline (Merck) containing 5% sucrose and concentrated to a desired volume.

Purified samples were subsequently analyzed by qPCR (to determine the vector genome copy number, GC concentration in crude lysate), SEC-HPLC (to determine the total amount of total AAV particles), FIX potency assay and infectivity assay in HelaRC32. Table 8 shows that the DuoDuoBac system (BacCapTrans1 + BacCapRep6) outperforms the comparable DuoBac system (BacCapRep6 + BacTrans4) at least in terms of vector yield, potency and infectivity.

TABLE 8

A comparison of various properties of AAV vectors produced with DuoBac or DuoDuoBac in 2 L tank bioreactors

| BacIDs | Infection ratios (%) | GC (E+11 gc/mL) | TP/GC ratio | Potency (RU) | Infectivity (gc/ip) |
|---|---|---|---|---|---|
| BacCapRep6 + BacTrans4 | 1 : 0.33 | 0.7 - 0.8 | 4.0-4.3 | 0.9 | 12.5 |
| BacCapTrans1 + BacCapRep6 | 0.33 : 0.33 | 2.6 - 2.7 | 8.9 - 9.3 | 1.1 | 28.1 |

### LITERATURE REFERENCES

1. Chaabihi, H., et al., Competition between baculovirus polyhedrin and p10 gene expression during infection of insect cells. J Virol, 1993. 67(5): p. 2664-71.
2. Hill-Perkins, M.S. and R.D. Possee, A baculovirus expression vector derived from the basic protein promoter of Autographa californica nuclear polyhedrosis virus. J Gen Virol, 1990. 71 ( Pt 4): p. 971-6.
3. Pullen, S.S. and P.D. Friesen, Early transcription of the ie-1 transregulator gene of Autographa californica nuclear polyhedrosis virus is regulated by DNA sequences within its 5′ noncoding leader region. J Virol, 1995. 69(1): p. 156-65.
4. Bosma, B., et al., Optimization of viral protein ratios for production of rAAV serotype 5 in the baculovirus system. Gene Ther, 2018. 25(6): p. 415-424.
5. Grieger, J.C., S. Snowdy, and R.J. Samulski, Separate basic region motifs within the adeno-associated virus capsid proteins are essential for infectivity and assembly. J Virol, 2006. 80(11): p. 5199-210.

1. A cell comprising one or more nucleic acid constructs, comprising:
(i) a first expression cassette comprising a first promoter operably linked to a nucleotide sequence encoding an mRNA, translation of which in the cell produces at least one of parvoviral Rep 78 and 68 proteins;
ii) a second expression cassette comprising a second promoter operably linked to a nucleotide sequence encoding an mRNA, translation of which in the cell produces at least one of parvoviral Rep 52 and 40 proteins;
(iii) a third expression cassette comprising a third promoter operably linked to a nucleotide sequence encoding parvoviral VP1, VP2, and VP3 capsid proteins; and,
(iv) a nucleotide sequence comprising a transgene that is flanked by at least one parvoviral inverted terminal repeat sequence, wherein,
at least one of the first and second expression cassette are present on a first nucleic acid construct with the third expression cassette, and
upon transfection of the cell with the one or more nucleic acid constructs, the first promoter is active before the second and third promoters.

2. The cell according to claim 1, wherein the nucleotide sequence comprising the transgene flanked by the parvoviral inverted terminal repeat sequence is present on a second nucleic acid construct.

3. The cell according to claim 2, wherein the second nucleic acid construct further comprises a fourth expression cassette comprising a fourth promoter operably linked to a nucleotide sequence encoding parvoviral VP1, VP2, and VP3 capsid proteins, wherein the first promoter is active before the second, third and fourth promoters, wherein optionally, the third and fourth promoters are identical, and wherein optionally, the parvoviral VP1, VP2, and VP3 capsid proteins encoded by the nucleotide sequences in the third and fourth expression cassettes are identical.

4. The cell according to claim 3, wherein the at least one of parvoviral Rep 78 and 68 proteins and the at least one of parvoviral Rep 52 and 40 proteins comprise a common amino acid sequence comprising the amino acid sequence from the second amino acid to the most C-terminal amino acid of the at least one of parvoviral Rep 52 and 40 proteins, wherein the common amino acid sequences of the at least one of parvoviral Rep 78 and 68 proteins and the at least one of parvoviral Rep 52 and 40 proteins are at least 90% identical, and wherein the nucleotide sequence encoding the common amino acid sequence of the at least one of parvoviral Rep 78 and 68 proteins and the nucleotide sequence encoding the common amino acid sequences of the at least one of parvoviral Rep 52 and 40 proteins are less than 90% identical.

5. The cell according to claim 4, wherein the common amino acid sequences of the at least one of parvoviral Rep 78 and 68 proteins and the at least one of parvoviral Rep 52 and 40 proteins are at least 99% identical.

6. The cell according to claim 4, wherein the nucleotide sequence encoding the common amino acid sequence of the at least one of parvoviral Rep 78 and 68 proteins has an improved codon usage bias for the cell as compared to the nucleotide sequence encoding the common amino acid sequences of the at least one of parvoviral Rep 52 and 40, or wherein the nucleotide sequence encoding the common amino acid sequence of the at least one of parvoviral Rep 52 and 40 proteins has an improved codon usage bias for the cell as compared to the nucleotide sequence encoding the common amino acid sequences of the at least one of parvoviral Rep 78 and 68 proteins.

7. The cell according to claim 6, wherein the difference in codon adaptation index between the nucleotide sequences coding for the common amino acid sequences in the at least one of parvoviral Rep 78 and 68 proteins and the at least one of parvoviral Rep 52 and 40 proteins is at least 0.2.

8. The cell according to claim 1, wherein the first promoter is a constitutive promoter.

9. The cell according to claim 1, wherein at least one of the second, third and fourth promoters is an inducible promoter.

10. The cell according to claim 9, wherein the inducible promoter is a viral promoter that is induced at least 24 hours after transfection or infection of the cell with the virus.

11. The cell according to claim 1, wherein at least one of the first and second nucleic acid construct is stably integrated in the genome of the cell.

12. The cell according to claim 1, wherein the cell is an insect cell, and wherein at least one the first and second nucleic acid construct is an insect cell-compatible vector.

13. The cell according to claim 12, wherein the insect cell-compatible vector is a baculoviral vector.

14. The cell according to claim 13, wherein:
(a) the first promoter is selected from a deltaEI promoter and an EI promoter; and,
(b) the second, third and fourth promoters are selected from a polH promoter and a p10 promoter.

15. 3The cell according to claim 13, wherein at least one expression cassette comprises at least one ecdysone responsive element and/or at least one baculovirus enhancer element selected from the group consisting of hr1, hr2, hr2.09, hr3, hr4, hr4b and hr5.

16. The cell according to claim 1, wherein the nucleotide sequence encoding an mRNA, translation of which in the cell produces only at least one of parvoviral Rep 78 and 68 proteins, comprises an intact parvoviral p19 promoter.

17. The cell according to claim 1, wherein the at least one of parvoviral Rep 78 and 68 proteins, the at least one of parvoviral Rep 52 and 40 proteins, the parvoviral VP1, VP2, and VP3 capsid proteins and the at least one parvoviral inverted terminal repeat sequence are from an adeno associated virus (AAV).

18. The cell according to claim 4, wherein the first nucleic acid construct is DuoBac CapRep6 (SEQ ID NO. 10) and the second nucleic acid construct is DuoBac CapTrans1 (SEQ ID NO. 12), and the first and second constructs are optionally present in a 3 : 1 molar ratio.

19. A method for producing a recombinant parvoviral virion in a cell, comprising:
(a) culturing a cell according to claim 1 under conditions such that recombinant parvoviral virion is produced; and,
(b) recovering the recombinant parvoviral virion.

20. The method according to claim 19, wherein the cell is an insect cell and/or the parvoviral virion is an AAV virion.

21. The method according to claim 19, wherein recovery of the recombinant parvoviral virion comprises at least one of affinity-purification of the virion using an immobilised anti-parvoviral antibody, and filtration over a filter having a nominal pore size of 30 - 70 nm.

22. A nucleic acid construct, comprising:
(i) a first expression cassette comprising a first promoter operably linked to a nucleotide sequence encoding an mRNA, translation of which in the cell produces at least one of parvoviral Rep 78 and 68 proteins;
ii) a second expression cassette comprising a second promoter operably linked to a nucleotide sequence encoding an mRNA, translation of which in the cell produces at least one of parvoviral Rep 52 and 40 proteins; and
(iii) a third expression cassette comprising a third promoter operably linked to a nucleotide sequence encoding parvoviral VP1, VP2, and VP3 capsid proteins.

23. A nucleic acid construct comprising a nucleotide sequence comprising a transgene that is flanked by at least one parvoviral inverted terminal repeat sequence.
